(19) United States
(12) Patent Application Publication
(10) Pub. No.: US 2006/0222657 A1
(43) Pub. Date: Oct. 5, 2006

(54) Polypeptide transduction and fusogenic peptides

(76) Inventors: Steven F. Dowdy, La Jolla, CA (US); Jehangir Wadia, La Jolla, CA (US)

Correspondence Address: BUCHANAN, INGERSOLL & ROONEY LLP, P.O. BOX 1404, ALEXANDRIA, VA, 22313-1404, US

(21) Appl. No.: 10/561,092

(22) PCT Filed: Jun. 18, 2004
(86) PCT No.: PCT/US04/20837

§ 371 Date: May 25, 2006

Related U.S. Application Data
(60) Provisional application No. 60/480,065, filed on Jun. 20, 2003.

(51) Int. Cl. A61K 39/21; A61K 39/12; C07K 14/16

(52) U.S. Cl. 424/186.1; 424/188.1; 530/350

(57) ABSTRACT

Due to the barrier imposed by the cell membrane, delivery of macromolecules in excess of 500 Daltons directly into cells remains problematic. However, proteins, which have been evolutionarily selected to perform specific functions, are therefore an attractive therapeutic agent to treat a variety of human diseases. In practice, the direct intracellular delivery of these proteins has, until recently, been difficult to achieve due primarily to the bioavailability barrier of the plasma membrane, which effectively prevents the uptake of the majority of peptides and proteins by limiting their passive entry. However, recent work using small cationic peptides, termed protein transduction domains (PTDs), derived from polynucleotide binding proteins, such as HIV TAT protein or the Drosophila transcription factor Antp. or synthetic poly-Arginine, have now been shown to deliver a myriad of molecules, including synthetic small molecules, peptides and proteins, into animal models in vivo.

### CROSS REFERENCE TO RELATED APPLICATIONS

This disclosure claims priority under 35 U.S.C. §119 to provisional application serial no. 60/480,065, filed Jun. 20, 2003, the disclosure of which is incorporated herein by reference.

### STATEMENT REGARDING FEDERALLY SPONSORED RESEARCH

This invention was funded in part by Grant No. CA96098 awarded by National Institutes of Health. The government may have certain rights in the invention.

### TECHNICAL FIELD

This disclosure relates to fusion polypeptides comprising a transduction moiety and a therapeutic or diagnostic moiety. More particularly the disclosure provides a composition comprising a plurality of fusion polypeptides, each comprising a transduction moiety and each individually comprising a fusogenic polypeptide or a heterologous polypeptide.

### BACKGROUND

Eukaryotic cells contain several thousand proteins, which have been, during the course of evolution, selected to play specific roles in the maintenance of virtually all cellular functions. Not surprisingly then, the viability of every cell, as well as the organism on the whole, is intimately dependent on the correct expression of these proteins. Factors which affect a particular protein's function, either by mutations or deletions in the amino acid sequence, or through changes in expression to cause overexpression or suppression of protein levels, invariably lead to alterations in normal cellular function. Such alterations often directly underlie a wide variety of genetic and acquired disorders. Consequently, the ability to manipulate cell biology at the protein level, without the use of DNA based methods, would provide a powerful tool for understanding and affecting complex biological processes and would likely be the basis for the treatment of a variety of human diseases. For instance, the reconstitution of tumor-suppressor function following the mutation or deletion of tumor-suppressor proteins, such as p53, in cancer therapy or the replacement of defective proteins in genetic disease such as cystic fibrosis or Duchenne's muscular dystrophy are often considered the goal of effective treatment (Anderson, W. Nature 392:25-30, 1998).

In practice however, the direct intracellular delivery of these proteins has been difficult. This is due primarily to the bioavailability barrier of the plasma membrane, which effectively prevents the uptake of the majority of peptides and proteins by limiting their passive entry.

Traditionally, approaches to modulate protein function have largely relied on the serendipitous discovery of specific drugs and small molecules which could be delivered easily into the cell. However, the usefulness of these pharmacological agents is limited by their tissue distribution and unlike “information-rich” proteins, they often suffer from poor target specificity, unwanted side-effects, and toxicity. Likewise, the development of molecular techniques for gene delivery and expression of proteins has provided for advances in our understanding of cellular processes but has been of little benefit for the management of genetic disorders (Robbins et al., Trends Biotechnol. 16:35-40, 1998; Robbins and Ghivizzani, Pharmacol. Ther. 80:35-47, 1998).

Apart from these gains however, the transfer of genetic material into eukaryotic cells either using viral vectors or by non-viral mechanisms such as microinjection, electroporation, or chemical transfection remains problematic. For instance, mammalian cells are frequently difficult to transfect, the expression of the target protein takes many hours to days to become detectable, the levels of protein expressed within each cell is highly variable and difficult to control, and there is significant toxicity associated with these transfection techniques. Moreover, in vivo gene therapy approaches using adenoviral vectors are associated with significant difficulties relating to a lack of target specificity and toxicity which have contributed to poor performance in several clinical trials (European Society of Gene Therapy, 2003; J. Gene Med. 5:82-84, 2003; Reid et al., Cancer Gene Ther. 9:979-86, 2002; Vile et al., Cancer Gene Ther. 9:1062-7, 2002).

### SUMMARY

The disclosure provides fusion polypeptides and compositions useful in cellular transduction and cellular modulation. The fusion polypeptides of the disclosure comprise a transduction moiety comprising a membrane transport function.

The disclosure provides a composition comprising a first fusion polypeptide comprising a first domain comprising a protein transduction moiety. The transduction moiety generally comprises a membrane transport function. The first fusion polypeptide further comprises a second domain comprising a heterologous polypeptide. The composition further comprises a second fusion polypeptide comprising a first domain comprising a protein transduction moiety, and a second domain comprising a fusogenic polypeptide.

The protein transduction moiety can be selected from a polypeptide comprising a herpesviral VP22 protein; a polypeptide comprising a human immunodeficiency virus (HIV) TAT protein; and a polypeptide comprising a homeodomain of an Antennapedia protein (Antp HD).

The heterologous polypeptide can be, for example, a therapeutic or diagnostic polypeptide such as an imaging agent. The therapeutic polypeptide can, for example, modulate cell proliferation by inhibiting or increasing cell proliferation. Further, the therapeutic agent can be a suicide inhibitor, such as thymidine kinase, or a tumor suppressor protein, such as p53.

An increase in cell proliferation can be obtained when the therapeutic agent is SV40 small T antigen, SV40 large T antigen, adenovirus E1A, papilloma virus E6, papilloma virus E7, Epstein-Barr virus, Epstein-Barr nuclear antigen-2, human T-cell leukemia virus-1 (HTLV-1), HTLV-1 tax, herpesvirus saimiri, mutant p53, myc, c-jun, c-ras, c-Ha-ras, h-ras, v-src, c-fgr, myb, c-myc, n-mye, v-myc, or Mdm2.

The disclosure further encompasses pharmaceutical or diagnostic compositions comprising the compositions described above. The disclosure also includes kits comprising a vessel or vessels containing a composition of the disclosure.

The disclosure further encompasses articles of manufacture comprising a vessel containing a first fusion polypeptide comprising a first domain comprising a protein transduction moiety, the transduction moiety comprising a membrane transport function; and a second domain comprising a heterologous polypeptide; and a second fusion polypeptide comprising a first domain comprising a protein transduction moiety, the transduction moiety comprising a membrane transport function; and a second domain comprising a fusogenic polypeptide; or packaged together, a vessel containing the aforedescribed polypeptides in separate vessels. The article of manufacture may further contain instructions for use of the composition in a therapeutic or diagnostic method.

The disclosure further encompasses methods of introducing a heterologous polypeptide in to a target cell, the method comprising contacting the cell with the composition of the disclosure.

The disclosure further encompasses methods of introducing a heterologous polypeptide in to a target cell, the method comprising contacting the cell with a composition comprising a first polypeptide comprising at least one transducing domain associated with a heterologous polypeptide; and a second polypeptide comprising at least one transducing domain associated with a fusogenic domain, wherein the first polypeptide and second polypeptide are co-transduced in to the cell. The contacting can be in vivo or in vitro.

The details of one or more embodiments are set forth in the accompanying drawings and the description below. Other features, objects, and advantages will be apparent from the description and drawings, and from the claims.

### BRIEF DESCRPIPTION OF THE FIGURES

FIG. 1 is a schematic diagram of the compositions and methods of the disclosure.

FIG. 2A shows a schematic diagram showing DNA recombination between loxP sites in tex.loxP.EG cells following treatment with TAT-Cre. The excision of the transcriptional stop region causes constitutive eGFP expression in recombined cells. Prior to analysis cells were incubated for 16-20 h following treatment in media containing serum to allow for sufficient expression of eGFP.

FIG. 2B shows a flow cytometry profiles of eGFP expression in untreated tex.loxP.EG cells or following treatment with 2 mM TAT-Cre or 2 mM Cre alone. Cells were incubated overnight in serum containing media and analyzed the following morning.

FIG. 2C is a time-course of TAT-Cre cellular uptake. Tex.loxP.EG cells were washed and replated into media with (□) or without (∘) serum and treated with 0.5 mM TAT-Cre. At each time point cells were washed by trypsinization.

FIG. 2D shows that extracellular GAG's prevent TAT-Cre recombination. Tex.loxP.EG cells were incubated for 1 h in serum free conditions with TAT-Cre and varying doses of either 0-50 mg/mL chondroitin sulfate A (□), B (∘), C (Δ) or 0-25 mg/mL heparin (∇).

FIG. 3A shows co-localization of TAT-Cre with endosomes. 3T3 cells were treated with 2 mM fluorescently labeled TAT-Cre-488 and 4 mM of the fluorescent endosomal marker FM 4-64 for 8 h.

FIG. 3B-C show recombination of tex.loxP.EG cells following TAT-Cre treatment is inhibited by lipid-raft destabilizing drugs. Cells were washed to remove serum and pretreated with 0-100 mg/mL nystatin (B) or 0-5 mM methyl-b-cyclodextrin (C) for 30′ prior to the addition of 0.1 mM (∘), 0.25 mM (□), 0.5 mM (⋄) TAT-Cre for 1 h.

FIG. 3D demonstrates the effect of nystatin on TAT-Cre internalization. Tex.loxP.EG cells were pre-incubated with nystatin for 30′ prior to the addition of TAT-Cre-488 and FM4-64. After 1 h, cells were trypsinized and washed prior to measurement of fluorescence by flow cytometry.

FIG. 4A shows that TAT-Cre does not co-localize with caveolin-1. NIH 3T3 cells were grown on a chambered coverglass and transfected with caveolin-1-gfp. Cells were then incubates with fluorescent TAT-CRE 546 for 1 h and corresponding images were captured. Higher magnification (insert) clearly shows cav-1-gfp and tat-cre 546 in different intracellular compartments.

FIG. 4B shows that lymphoid cells do not express caveolin-1 protein. Cell lysates from endothelial cells (EC), tex.loxP.EG cells (MTL), Jurkat T cells, and NIH 3T3 cells were blotted for cav-1 expression.

FIG. 4C-D shows that the inhibition of macropinocytosis prevents TAT-Cre mediated recombination. Tex.loxP.EG cells were pre-incubated with either 0-5 mM amiloride or 0-10 mM cytochalasin D before addition of increasing concentrations of 0.1 mM (∘), 0.25 mM (□), 0.5 mM (⋄) TAT-Cre for 1 h. Both amiloride (C) and cytochalasin D (D) causes a dose-dependent decrease in recombination.

FIG. 5A shows that chloroquine increases TAT-Cre recombination. Equal numbers of 3T3 loxP.lacZ cells were treated with 0.25 mM TAT-Cre with 0-200 mM chloroquine overnight in DMEM +10% serum. The following day, recombination and lacZ expression was measured by in situ β-galactosidase staining.

FIG. 5B-C shows the efficiency of TAT-Cre recombination is enhanced by HA2-TAT induced endosomal release. Tex.loxP.EG cells were treated with TAT-Cre and either 0 mM (□), 1 mM (∘), 2.5 mM (Δ), or 5 mM HA2-TAT (∇) peptide overnight in RPMI +10% serum. The next day eGFP expression was measured by flow cytometry.

FIG. 5D shows nystatin pretreatment blocks the effect of HA2-TAT peptide. Tex.loxP.EG cells were pretreated with nystatin for 30′ in serum-free media after which either 0.1 mM (▪, □) or 0.25 mM (●, ∘) TAT-Cre +/− 5 mM HA2-TAT was added for 1 h. Cells were then washed and replated overnight in normal media.

FIG. 6 shows the pTAT 2.1 plasmid map and sequence.

FIG. 7 shows the pTAT 2.2 plasmid map and sequence.

FIG. 8 shows the pTAT 2.2 CRE plasmid map and sequence.

### DETAILED DESCRIPTION

As used herein and in the appended claims, the singular forms “a,” “and,” and “the” include plural referents unless the context clearly dictates otherwise. Thus, for example, reference to “a target cell” includes a plurality of such cells and reference to “the expression vector” includes reference to one or more transformation vectors and equivalents thereof known to those skilled in the art, and so forth.

Unless defined otherwise, all technical and scientific terms used herein have the same meaning as commonly understood to one of ordinary skill in the art to which this disclosure belongs. Although any methods, cells and genes similar or equivalent to those described herein can be used in the practice or testing of the disclosed methods and compositions, the exemplary methods, devices and materials are now described.

All publications mentioned herein are incorporated herein by reference in full for the purpose of describing and disclosing the cell lines, vectors, and methodologies which are described in the publications which might be used in connection with the description herein. The publications discussed above and throughout the text are provided solely for their disclosure prior to the filing date of the present application. Nothing herein is to be construed as an admission that the inventors are not entitled to antedate such disclosure by virtue of prior disclosure.

An advantage of protein transduction is the intracellular delivery of proteins which are otherwise difficult to transfect and where microinjection is not a possible option. For instance, primary lymphocytes are very difficult to transfect, requiring electroporation of DNA constructs. This process very inefficient, killing 90-99% of the cells, and yielding protein expression in less than 10% of those which survive.

The ability to deliver full-length functional proteins into cells is problematical due to the bioavailability restriction imposed by the cell membrane. That is, the plasma membrane of the cell forms an effective barrier which restricts the intracellular uptake of molecules to those which are sufficiently non-polar and smaller than approximately 500 daltons in size. Previous efforts to enhance the internalization of proteins have focused on fusing proteins with receptor ligands (Ng et al., Proc. Natl. Acad. Sci. USA, 99:10706-11, 2002) or by packaging them into caged liposomal carriers (Abu-Amer et al., J. Biol. Chem. 276:30499-503, 2001). However, these techniques often result in poor cellular uptake and intracellular sequestration into the endocytic pathway.

The disclosure provides fusion polypeptides and compositions useful in cellular transduction and cellular modulation. The fusion polypeptides of the disclosure comprise a transduction moiety comprising a membrane transport function. Transduction domains comprising cationic moieties have been used for transduction of cells. However, the delivery of such fusion protein through the cell membrane is only one part of the process of transduction. A subsequence process is the release of the fusion protein out of the endocytic vesicles and into the cytoplasm, nucleus of other organelle. For example, once TAT-fusion proteins are taken into a cell by endocytosis they remain bound within intracellular vesicles. Thus, the later process of delivery into the cytoplasm, nucleus or organelle does not occur timely or efficiently.

The recent discovery of several proteins which could efficiently pass through the plasma membrane of eukaryotic cells has led to the identification of a novel class of proteins from which peptide transduction domains have been derived. The best characterized of these proteins are the Drosophila homeoprotein antennapedia transcription protein (AntHD) (Joliot et al., New Biol. 3:1121-34, 1991; Joliot et al., Proc. Natl. Acad. Sci. USA, 88:1864-8, 1991; Le Roux et al., Proc. Natl. Acad. Sci. USA, 90:9120-4, 1993), the herpes simplex virus structural protein VP22 (Elliott and O'Hare, Cell 88:223-33, 1997) and the HIV-1 transcriptional activator TAT protein (Green and Loewenstein, Cell 55:1179-1188, 1988; Frankel and Pabo, Cell 55:1189-1193, 1988). Not only can these proteins pass through the plasma membrane but the attachment of other proteins, such as the enzyme β-galactosidase, was sufficient to stimulate the cellular uptake of these complexes. Such chimeric proteins are present in a biologically active form within the cytoplasm and nucleus. Characterization of this process has shown that the uptake of these fusion polypeptides is rapid, often occurring within minutes, in a receptor independent fashion. Moreover, the transduction of these proteins does not appear to be affected by cell type and can efficiently transduce 100% of cells in culture with no apparent toxicity (Nagahara et al., Nat. Med. 4:1449-52, 1998). In addition to full-length proteins, protein transduction domains have also been used successfully to induce the intracellular uptake of DNA (Abu-Amer, supra), antisense oligonucleotides (Astriab-Fisher et al., Pharm. Res, 19:744-54, 2002), small molecules (Polyakov et al., Bioconjug. Chem. 11:762-71, 2000) and even inorganic 40 nanometer iron particles (Dodd et al., J. Immunol. Methods 256:89-105, 2001; Wunderbaldinger et al., Bioconjug. Chem. 13:264-8, 2002; Lewin et al., Nat. Biotechnol. 18:410-4, 2000; Josephson et al., Bioconjug., Chem. 10:186-91, 1999) suggesting that there is no apparent size restriction to this process.

The fusion of a protein transduction domain (PTD) with a heterologous molecule (e.g., a polynucleotide, small molecule, or protein) is sufficient to cause their transduction into a variety of different cells in a concentration-dependent manner. Moreover, this technique for protein delivery appears to circumvent many problems associated with DNA and drug based techniques. This technique represents the next paradigm in the ability to modulate cells and offer a unique avenue for the treatment of disease.

PTDs are typically cationic in nature. These cationic protein transduction domains track into lipid raft endosomes and release their cargo into the cytoplasm by disruption of the endosomal vesicle. Examples of PTDs include AntHD, TAT, VP22, and functional fragments thereof. The disclosure provides methods and compositions that combine the use of PTDs such as TAT and poly-Arg, with a fusogenic, transducible peptide (e.g., HA2-TAT) to enhance transduction into cells in a non-toxic fashion in lipid raft endosomes.

Cationic TAT and poly-Arg protein transduction domains can deliver biologically active “cargo” into mammalian cells. The methods are useful for the treatment of a number of diseases and disorders including, but not limited to, stroke, psoriasis and cancer. Using a transducible TAT-Cre recombinase reporter protein, it was determined that transduction occurs by an initial ionic cell surface interaction, followed by a cholesterol, lipid-raft mediated endocytosis. Based on the mechanism of transduction, a transducible influenza fusogenic HA2-TAT peptide was developed that enhanced the transduction efficiency of TAT-Cre greater than ten-fold in the absence of cytotoxicity. The gene therapy world has used endosomal disruptors, such as such as chloroquine and PEI, to enhance gene therapy. However, these generalized endosomal disruptors cause significant cytotoxicity and cell death. In contrast, endosomal disrupters, such as chloroquine and PEI, moderately increased transduction efficiency, but caused extensive cytotoxicity. The combination of a transducible and fusogenic peptide (e.g., TAT-HA2) is unique.

In general, the transduction domain of the fusion molecule can be nearly any synthetic or naturally-occurring amino acid sequence that can transduce or assist in the transduction of the fusion molecule. For example, transduction can be achieved by use of a polypeptide comprising a PTD (e.g., an HIV TAT protein or fragment thereof) that is covaleritly linked to a fusogenic molecule. Alternatively, the transducing protein can be the Antennapedia homeodomairi or the HSV VP22 polypeptide, or suitable transducing fragments thereof.

The type and size of the PTD will be guided by several parameters including the extent of transduction desired. Typical PTDs will be capable of transducing at least about 20%, 25%, 50%, 75%, 80% or 90% of the cells of interest, more typically at least about 95%, 98% and up to and including about 100% of the cells. Transduction efficiency, typically expressed as the percentage of transduced cells, can be determined by several conventional methods such as flow cytometric analysis.

PTDs will typically manifest cell entry and exit rates that favor at least picomolar amounts of the fusion molecule in the cell. The entry and exit rates of the PTDs can be readily determined or at least approximated by standard kinetic analysis using detectably-labeled fusion molecules.

Additionally provided are chimeric PTDs that include parts of at least two different transducing proteins. For example, chimeric PTDs can be formed by fusing two different TAT fragments, e.g., one from HIV-l and the other from HIV-2.

PTDs can be linked or fused with any number of heterologous molecules that provide diagnostic utility and/or therapeutic utility. As used herein, a heterologous molecule can be (1) any heterologous polypeptide, or fragment thereof, (2) any polynucleotide (e.g., a ribozyme, antisense molecule, polynucleotide, oligonucleotide and the Like); and (3) any small molecule, that is capable of being linked or fused to a PTD. For example, PTD fusion molecule can comprise a PTD linked to a heterologous polypeptide, or fragment thereof, that provides a therapeutic effect when present in a targeted cell. The term “therapeutic” is used in a generic sense and includes treating agents, prophylactic agents, and replacement agents. Examples of therapeutic molecules include, but are not limited to, cell cycle control agents; agents which inhibit cyclin proteins, such as antisense polynucleotides to the cyclin G1 and cyclin D1 genes; growth factors such as, for example, epidermal growth factor (EGF), vascular endothelial growth factor (VEGF), erythropoietin, G-CSF, GM-CSF, TGF-α, TGF-β, and fibroblast growth factor; cytokines, including, but not limited to, Interleukins 1 through 13 and tumor necrosis factors; anticoagulants, anti-platelet agents; anti-inflammatory agents; tumor suppressor proteins; clotting factors including Factor VIII and Factor IX, protein S, protein C, antithrombin III, von Willebrand Factor, cystic fibrosis transmembrane conductance regulator (CFTR), and negative selective markers such as Herpes Simplex Virus thymidine kinase.

In addition, a heterologous molecule fused to the PTD can be a negative selective marker or “suicide” protein, such as, for example, the Herpes Simplex Virus thymidine kinase (TK). Such a PTD linked to a suicide protein may be administered to a subject whereby tumor cells are transduced. After the tumor cells are transduced with the kinase, an interaction agent, such as gancyclovir or acyclovir, is administered to the subject, whereby the transduced tumor cells are killed. Growth of the tumor cells is inhibited, suppressed, or destroyed upon expression of the anti-tumor agent by the transduced tumor cells.

In addition, a heterologous molecule can be an imaging agent. Thus, it is to be understood that the disclosure is not to be limited to the diagnosis and treatment of any particular disease or disorder.

The disclosure provides methods and compositions that enhance uptake and release of PTDs linked to such heterologous molecules. A PTD fusion polypeptide comprising a PTD domain and fusogenic domain enhances the release of the PTD-heterologous fusion polypeptide.

The transducible PTD-fusogenic fusion polypeptide (e.g., HA2-TAT fusion polypeptide) enhances release of heterologous molecules from the endosome into the cytoplasm, nucleus or other cellular organelle. This is accomplished by the PTD-fusogenic fusion polypeptide tracking with the TAT-heterologous fusion polypeptide via independent or the same PTD domain and then fusing to the vesicle lipid bilayer by the fusogenic domain (e.g., HA2) resulting in an enhanced release into the cytoplasm, nucleus, or other cellular organelle. Thus, the disclosure provides a transduction domain (PTD) associated with a heterologous molecule and a transduction domain (PTD) associated with a fusogenic (i.e., facilitates membrane fusion) domain. For example, a PTD associated with a heterologous molecule can comprise a single chimeric/fusion polypeptide. Similarly, a PTD associated with a fusogenic domain can comprise a single chimeric/fusion polypeptide. The fusion of functionally distinguishable domains to generate chimeric/fusion polypeptides is known in the art. The direct delivery and efficient cellular uptake of transducing proteins is an exciting new tool which offers several advantages over traditional DNA-based methods for manipulating the cellular phenotype.

The advantages and versatility of protein transduction over viral transgene delivery were studied. In contrast to viral transduction, which had limited capacity to infect non-dividing cells, all cell types were susceptible to TAT-mediated transduction. Moreover, with protein transduction-mediated delivery, it was possible to achieve equal cellular concentrations of TAT-β-galactosidase in 100% of the cells in contrast to viral delivery, which could achieve only 30-50% transduction efficiency with highly variable levels of expression within those cells. Furthermore, β-galactosidase activity could be readily detected intracellularly within ascinar cells, as early as 10 minutes following tissue injection, and up to 6 hours following, while viral delivery was associated with a significantly delayed onset of enzyme activity due to the added cellular requirement for the transcription and translation of the protein.

The HIV-1 TAT protein is an essential viral regulatory factor which is involved in the trans-activation of genes involved in the HIV long terminal repeat and therefore plays an essential role in viral replication (Sodroski et al., Science 227:171-173, 1985). Full length TAT protein is encoded by two exons and is between 86 and 102 amino acids in length depending on the strain of virus. It is organized into three functional domains consisting of: (1) an N-terminal acidic region involved in trans-activation, (2) a cysteine-rich DNA binding region with a zinc-finger motif and, (3) a basic region which is thought to be required for nuclear import. In 1988 two groups which were independently studying the trans-activating properties of HIV-1 TAT protein (Green and Loewnstein, Cell 55:1179-1188, 1988; and Frankel and Pabo, Cell 55:1189-1193, 1988), described a surprising property of this protein; exogenously added TAT protein could transactivate the viral promoter within cells in culture. Recombinant TAT protein, in the absence of any external perturbations, when added to the culture media was sufficient to induce reporter activity at concentrations as low as 1 nM (Frankel and Pabo, supra). Other cell lines including Jurkat T cells, H9 lymphocytic and U937 promonocytic cells were subsequently found to internalize TAT protein. Green and Loewenstein, also studying the trans-act vation of TAT in HeLa cells using DNA transfection and protein microinjection, found that chemically-synthesized TAT-86 was rapidly internalized into cells in culture and could trans-activate the expression of the reporter (Green and Loewenstein, supra). These experiments demonstrated for the first time a novel biological phenomenon in which a large, full-length, protein could be added exogenously to cells in culture and rapidly internalized in an apparent receptor-less mechanism. Although tat-fusion proteins are taken into the cell by endocytosis they remain bound within intracellular vesicles. Thus, the full use of the fusion constructs does not occur timely or efficiently.

To measure the time-course of TAT transduction, cells were treated with full-length TAT protein for different intervals of time (Mann and Frankel, EMBO J. 10:1733-1739, 1991). Surprisingly, in all cell types used, the maximal increase in biological activity occurred after 5 minutes of treatment. Using radio-iodinated TAT, approximately 50% of the protein was found bound to the plasma membrane by 1 min. at 37° C. and 80% bound after 15 minutes; incubation with cells at low temperature did not affect the rate of binding in these experiments. Further characterization by Feinberg et al. examining reporter MRNA levels showed that TAT-activation could be detected after 15 minutes of incubation and reached a maximum after 2 hours, further supporting the observation that internalization of protein was rapid (Feinberg et al., Proc. Natl. Acad. Sci. USA 88:4045-9, 1991). In an attempt to determine the affinity and number of binding sites involved in the uptake of TAT protein, endocytosis of labeled TAT in HeLa and H9 cells was measured. Binding of TAT to the cell membrane did not involve any specific receptors, was not affected by low temperature, and was only saturable at very high protein concentrations (Mann and Frankel, supra). The lack of specific receptor required for entry of TAT was further demonstrated when pretreatment of the cells with trypsin, to digest membrane spanning receptors, prior to the addition of TAT protein could not block reporter trans-activation.

Furthermore, the removal of sialic acid and heparin from the cell surface similarly had no effect, suggesting that charged polysaccharides on the cell surface did not participate in TAT binding. However, since the intracellular accumulation of TAT can be competitively blocked by increasing concentrations of polyanions, such as heparin and dextran sulphate, or by using a mAb against the basic region of TAT, the nature of the initial binding of TAT to the cell surface may still involve interactions with positively charged molecules (Tyagi et al., J. Biol., Chem., 276:3254-61, 2001; Hankansson et al., Protein Sci. 10:2138-9, 2001).

TAT-mediated protein transduction has demonstrated that large proteins such as β-galactosidase, horseradish peroxidase and RNAase A can be transduced into cells by chemically cross-linking them to peptides corresponding to amino acids 1-72 or 37-72 of TAT (SEQ ID NO:1) (Fawell et al., PNAS, 91:664-668, 1994). These TAT-conjugates were predominantly found associated on the cell surface by 20 minutes followed by a progressive intracellular accumulation over the next 6 hours with little difference between TAT peptide fusions consisting of amino acids 1-72 or 37-72 (SEQ ID NO:1). After overnight incubation with TAT-β-galactosidase, trypsin sensitive and insensitive activities were determined to separate surface bound from internalized protein. Approximately 5×10⁶ molecules were associated with each cell, 20 percent of which were trypsin-insensitive indicating the full internalization of the protein.

Significantly, all the cells in culture showed uptake of the TAT protein and transduction of TAT-β-galactosidase could be achieved in all cell types which were tested including HeLa, COS-1, CHO, H9, NIH 3T3, primary human keratinocytes, and umbilical endothelial cells. Interestingly, unlike TAT activation of the HIV-1 LTR following transduction which was increased by the addition of chloroquine, quantitative analysis of TAT-β-galactosidase activity showed less than a two fold increase following treatment with various endo-osmotic agents (Fawell et al., supra). However, since β-galactosidase activity could be recovered from within endosomes following fixation and staining, it was not possible to determine how much of the protein was in the cytoplasm in this way. To address this question a functional assay using a conjugate of TAT-RNAase A was tested for its ability to inhibit protein synthesis through the nonspecific degradation of cellular RNA. In this model, if TAT-RNAase A were entering the cell only by endocytosis there should be no effect on protein synthesis. However, addition of TAT-RNAase A was sufficient to decrease cellular protein synthesis and induce toxicity at high doses indicating the presence of protein within the cytoplasm.

While it had been conclusively shown that chemical conjugates of heterologous full length proteins with the TAT 37-72 peptide could be effectively delivered through the plasma membrane of cells, Vivès et al. characterized shorter domains of the TAT protein which were sufficient for cell internalization in an effort to improve the cellular uptake and activity of conjugated proteins (Vivès et al., JBC, 272:16010-16017, 1997).

Starting with a peptide encompassing residues 37-60 of TAT (SEQ ID NO:1) which included both the basic region and the putative α-helical domain, a series of truncations at either the C or N terminal were constructed. In this way two fragments containing the entire basic region, TAT-(43-60) (LGISYGRKKRRQRRRPPQ; SEQ ID NO:1 from amino acid 43-60) and TAT-(48-60) (GRKKRRQ RRRPPQ; SEQ ID NO:1 from amino acid 48-60), but with deletions in the α-helical domain fully retained the ability for cell internalization and nuclear localization while TAT-(37-53) (FITKALGISYGRKKRR; SEQ ID NO:1 from amino acid 37-53), which had a 7 amino acid deletion in the basic region but retained the α-helical structure was not able to transduce into cells, even at high concentrations. In addition, the short 13 residue TAT-(48-60) peptide appeared to be more efficiently transduced than other active peptide sequences indicating that the ordered secondary structure provided by the α-helical region was not necessary for transduction. Truncation of the C-terminal Pro-Pro-G1n from TAT-(48-60) further characterized the minimal transduction domain to consist of amino acids 47-57 (YGRKKRRQRRR; SEQ ID NO:1 from amino acid 47-57). The transduction of the TAT basic peptide did not involve any disruption of the plasma membrane and could not promote the uptake of unrelated non-conjugated peptides or molecules indicating that the mechanism of transduction was highly specific.

Since the initial discovery of TAT transduction, novel transduction domains have been identified within several other proteins including antennapedia protein (Perez et al., (1992) J. Cell Sci. 102 ( Pt 4), 717-22, Fujimoto et al., (2000) Cancer Lett. 159, 151-8, Thoren et al., (2000) FEBS Lett. 482, 265-8) and VP22 protein (Phelan et al., Nat. Biotechnol. 16:440-3, 1998; Elliott et al., Gene Ther., 6:149-51, 1999; Brewis et al., J. Virol., 74:1051-6, 2000), as well as synthetic peptoid carriers such as poly-arginine (Uemura et al., Circ. J. 66:1155-60, 2002; Wender et al., J. Am. Chem. Soc. 124:13382-3, 2002; Rothbard et al., J. Med. Chem. 45:3612-8, 2002). Although there does not appear to be any homology between the primary and secondary structure of these protein transduction domains, the rate of cellular uptake has been found to strongly correlate to the number of basic residues present, indicating the presence of a common, internalization mechanism which is likely dependent on an interaction between the charged side groups of the basic residues and lipid phosphates on the cell surface (Futaki et al., J. Biol., Chem. 276:5836-40, 2001; Wender et al., Proc. Natl. Acad. Sci. USA 97:13003-8, 2000).

While these different protein transduction domains show similar characteristics for cellular uptake, they vary in their efficacy for transporting protein cargo into cells. To date, fusion polypeptides created with a PTD comprising TAT-(47-57) have shown markedly better cellular uptake than similar fusions using the 16 amino acid sequence from antennapedia or VP22, although recently devised peptide sequences such as the retro-inverso form of TAT-(57-47) or homopolymers of arginine appear to increase cellular uptake several-fold (Futaki et al., supra; Wender et al., supra). For example, the antennapedia protein transduction domain can transduce into cells when associated with chemically synthesized peptides; however, the efficiency dramatically decreases with the incorporation of larger proteins (Kato et al., FEBS Lett. 427:203-8, 1998; Chen et al., Proc. Natl. Acad. Sci. USA 96:4325-9, 1999). VP22 transduction is somewhat different from TAT or antennapedia peptide, requiring the DNA encoding the entire VP22 protein to be cloned to the gene of interest and then transfected into cells. The translated fusion polypeptide then transduces from the primary transfected cells into the surrounding cells at varying levels (Elliott and O'Hare, Cell 88:223-33, 1997; Elliott and O'Hare, Gene Ther. 6:149-51, 1999).

A large variety of full length TAT fusion polypeptides of 15 to 121 kDa in size and spanning a wide variety of functional classes such as cell cycle proteins, DNA modifying enzymes, signaling proteins, and anti-apoptotic proteins have been purified and shown to be effectively delivered into cells with biological activity. A few examples of these include TAT-p16, TAT-p27 (Nagahara et al., supra), adenovirus TAT-E1A, TAT-HPV E7, TAT-caspase-3 (Vocero-Akbani et al., Nat. Med. 5:29-33, 1999), TAT-HIV protease (Id.), TAT-Bid, TAT-eGFP (Caron et al., Mol., Ther. 3:310-8, 2001), TAT-Ikβ, TAT-Rho, TAT-Rac, TATCDC42, TAT-Cdk2 dominant-negative, TAT-cre (Joshi et al., Genesis. 33:48-54, 2002; Peitz et al., Proc. Natl. Acad. Sci. USA 99:4489-94, 2002), TAT-p73 dominant-negative (Lissy et al., Immunity *:57-65, 1998), TAT-E2F-1 dominant-negative (Lissy et al., Nature 407:642-5, 2000) and TAT-pRb. In vitro both primary and transformed cell types including peripheral blood lymphocytes, diploid human fibroblasts, keratinocytes, bone marrow stem cells, osteoclasts, fibrosarcoma cells, leukemic T cells, osteosarcoma, glioma, hepatocellular carcinoma, renal carcinoma and NIH3T3 cells have been transduced with recombinant TAT-proteins.

In the past several years a wide variety of full-length proteins and peptides have been successfully transduced into cells both in vitro and in vivo by fusion with the TAT protein transduction domain (Table 1). These applications cover a broad range of uses and, in general, there appears to be no particular limitation in either the size or type of protein that can be delivered. TAT protein transduction has been useful in a variety of situations to overcome the limitations of traditional DNA-based approaches or for the development of novel strategies in the treatment of disease.

TABLE 1

| TAT-Protein | Effect | References |
|---|---|---|
| TAT-Bcl-xL | anti-apoptotic | Cao et al-, |
| | | (2002) |
| | | J. Neurosci. |
| | | 22, 5423-31, |
| | | Kilic et |
| | | al., (2002) |
| | | Ann. Neurol. |
| | | 52, 617-22, |
| | | Dietz et |
| | | al., (2002) |
| | | Mol. Cell |
| | | Neurosci. |
| | | 21, 29-37, |
| | | Embury et |
| | | al., (2001) |
| | | Diabetes 50, |
| | | 1706-13 |
| TAT-p53 | tumor suppressor protein | Takenobu et |
| | | al., (2002) |
| | | Mol. Cancer |
| | | Ther. 1, |
| | | 1043-9 |
| TAT-ARC | transduction into myocardium is | Gustafsson |
| | cardioprotective | et al., |
| | | (2002) |
| | | Circulation |
| | | 106, 735-9 |
| TAT-cyclin | E restoration of proliferation | Hsia et al., |
| | | (2002) Int. |
| | | Immunol. 14, |
| | | 905-16 |
| TAT-glutamate | restoration of GDH-deficiency | Yoon et al., |
| dehydrogenase | disorders | (2002) |
| | | Neurochem- |
| | | Int. 41, 37-42 |
| TAT-Cu, Zn-SOD | antioxidant protein | Kwon et al., |
| | | (2000) FEBS |
| | | Lett. 485, |
| | | 163-7, Eurn |
| | | et al., |
| | | (2002) Mol. |
| | | Cells 13, |
| | | 334-40 |
| TAT-catalase | antioxidant protein | Jin et al-, |
| | | (2001) Free |
| | | Radic. Biol. |
| | | Med. 31, |
| | | 1509-19 |
| TAT-ODD-Caspase 3 | anti-tumor activity | Harada et |
| | | al., 2002) |
| | | Cancer Res. |
| | | 62, 2013-8 |
| TAT-HIV1-Caspase 3 | specific killing of HIV-infected | Vocero- |
| | cells | Akbani et |
| | | al., (1999) |
| | | Nat. Med. 5, |
| | | 29-33 |
| TAT-Cre | site-specific recombination | Joshi et |
| | | al., (2002) |
| | | Genesis. 33, |
| | | 48-54, Peitz |
| | | et al., |
| | | (2002) Proc. |
| | | Natl. Acad. |
| | | Sci. USA 99, |
| | | 4489-94 |
| TAT-APOBEC | editing of ApoB mRNA | Yang et al., |
| | | (2002) Mol. |
| | | Pharmacol. |
| | | 61, 269-76 |
| TAT-GFP | fluorescent protein | Caron et |
| | | al., (2001) |
| | | Mol. Ther. |
| | | 3, 310-8, |
| | | Han et al., |
| | | (2001) Mol. |
| | | Cells 12, |
| | | 267-71 |
| TAT-H-Ras | cytoskeletal reorganization | Hall et al., |
| | | (2001) Blood |
| | | 98, 2014-21 |
| TAT-IkappaB | NF-kappaB inhibitory protein | Abu-Amer et |
| | | al., 2001) |
| | | J. Biol. |
| | | Chem. 276, |
| | | 30499-503. |
| TAT-HPC-1/syntaxin | inhibitor of neurotransmitter | Fujiwara et |
| | release | al., (2001) |
| | | Biochim. |
| | | Biophys. |
| | | Acta 1539, |
| | | 225-32 |
| TAT-p16 | inhibitor of cyclin D/cdk | Ezhevsky et |
| | complexes | al., (2001) |
| | | Mol. Cell |
| | | Biol. 21, |
| | | 4773-84 |
| TAT-p27 | cyclin-dependent kinase | McAllister |
| | inhibitor | et al., |
| | | (2003) Mol. |
| | | Cell Biol. |
| | | 23, 216-28 |
| TAT-b-galactosidase | frequently used reporter enzyme | Barka et |
| | | al., (2000) |
| | | J. Histochem. |
| | | Cytochem. |
| | | 48, 1453-1460, |
| | | Schwarze et |
| | | al., (1999) |
| | | Science 285, |
| | | 1569-72 |
| TAT-p21 | cell cycle arrest in G1 phase | Kunieda et |
| | | al., (2002) |
| | | Cell |
| | | Transplant |
| | | 11, 421-8 |
| TAT-PEA-15 | prevents apoptosis by TNFa in | Embury et |
| | pancreatic cell line | al., (2001) |
| | | Diabetes 50, |
| | | 1706-13 |
| TAT-beta- | lysosomal enzyme | Xia et al., |
| glucuronidase | | (2001) Nat. |
| | | Biotechnol. |
| | | 19, 640-4 |

Protein transduction has been used effectively for studying the biology of several proteins. For instance, small GTPases, such as cdc42, rac, and rho, regulate the cytoskeletal architecture of the cell depending on the type of extracellular signals received (Zhong et al., Mol. Biol. Cell. 8:2329-44, 1997; Barry et al., Cell Adhes. Commun. 4:387-98, 1997). However, dissecting the role of these proteins in cytoskeletal remodeling in osteoclasts has been hampered by an inability to manipulate these cells since they are essentially resistant to the introduction of expression constructs by transfection or retroviral infection. In this case, the use of TAT-mediated transduction has allowed this restriction to be overcome.

Constitutively active and dominant-negative forms of TAT-rho protein were generated and added to osteoclast cultures resulting in the uptake of these proteins into 90-100% of cells, as measured by confocal microscopy. Within minutes after application, the constitutively active TAT-rho-V14 stimulated the formation of actin stress fibers in a manner indistinguishable from the growth factor osteopontin while dominant-negative TAT-rho was sufficient to block the effects of osteopontin. By using TAT-protein transduction, these experiments were able to demonstrate that integrin-dependent activation of phosphoinositide synthesis, actin stress fiber formation, podosome reorganization for osteoclast motility, and bone resorption all require rho stimulation.

Cre recombinase is a 38 kDa protein from bacteriophage P1 which mediates the site-specific, intramolecular or intermolecular recombination of DNA, between pairs of 13 bp inverted repeat sequences called loxP sites, permitting the precise deletion or incorporation of genes. Cre recombinase is increasingly being used to study biological phenomenon following the conditional knock-out or knockin of genes in vitro and in vivo but is hampered by the inefficiency of transfection and the limited number of transgenic mouse lines that express recombinase in appropriate cell types. The ability to target 100% of cells by TAT transduction and control cre-mediated recombination by cell-permeable recombinase has led to the development of transducible cre (Joshi et al., supra; Lissy et al., supra). In one application, TAT-cre was used on primary splenocytes harvested from retinoblastoma loxP mice to cause the site-specific excision of exon 19 from the retinoblastoma gene. After overnight incubation, PCR analysis and subsequent sequencing of the exon 19 region showed that predominantly all cells in culture contained the specific exon 19 deletion while cells treated with recombinant cre alone were not affected. Moreover, these results could be reproduced in vivo following intraperitoneal administration of TAT-cre and was only limited by proteolytic degradation of the protein by serum proteases. Similarly, TAT-cre has been shown to induce greater that 95% recombination efficiency in fibroblasts and murine embryonic stem cells in vitro (Joshi et al., supra; Peitz et al., supra). Moreover, transducible cre, utilizing a transduction domain identified from Karposi fibroblast growth factor, has been used to enzymatically recombine the majority of tissues following intraperitoneal administration in mice (Jo et al., Nat. Biotechnol. 19:929-33, 2001).

Intraperitoneal delivery of 200-500 mg of TAT-β-galactosidase, equivalent to 10-25 mg/kg of body weight of protein, into mice resulted in readily detectable β-galactosidase enzymatic activity in the majority of tissues assayed 4 h after injection (Schwarze et al., Science, 285:1596-72, 1999). β-galactosidase activity was strongest in the liver, kidney, lung, heart and spleen and significantly was found to cross through the blood-brain barrier and enter cells in the brain. TAT-β-galactosidase transduction did not disrupt the blood-brain barrier nor cause any observable disorders in the mouse.

Therefore, after demonstrating the introduction of a 120 kDa enzyme into many, if not all, cells and tissues in vivo it may now be possible to use a similar approach to combat inherited diseases by replacing malfunctioning or missing proteins or to specifically modulate cellular function by the specific introduction of novel proteins.

Solid tumors often contain significant areas of hypoxia which are more likely to be resistant to conventional radiotherapy and chemotherapy. The tumor's response to hypoxia is mediated by activation of the transcription factor HIF-1a, which causes the up-regulation of a variety of factors responsible for solid tumor expansion Ryan et al., 1998) EMBO J. 17, 3005-15. Interestingly, the regulation of HIF-1a occurs through an increase in its half-life in response to hypoxia Yu et al., (1998) Am. J. Physiol. 275, L818-26.

A 200 amino acid oxygen dependent degradation (ODD) domain within HIF-1a was identified and shown to control the protein's degradation, in the absence of hypoxia signaling, by the ubiquitin-proteosome pathway Huang et al., (1998) Proc. Natl. Acad. Sci. USA 95, 7987-92. By utilizing the properties of the ODD domain, Harada et al., have devised a novel cancer therapy based on a TAT-ODD-caspase 3 fusion polypeptide to induce cell death within the hypoxic regions of tumors Harada et al., 2002) Cancer Res. 62, 2013-8. When this TAT protein was injected intraperitoneally into tumor bearing mice the active protein was found to be stabilized in the solid tumors and not present throughout the normal tissues. Significantly, the administration of TAT-ODD-caspase-3 wild type, but not an inactive mutant of caspase-3, was able to suppress tumor growth and reduce the tumor mass after a single administration without obvious side-effects.

In one such example, TAT-antigen transduction was used to induce the expression of defined tumor antigens on dendritic cells and generate cytotoxic T lymphocyte responses, circumventing the limitations of transfection and the concerns surrounding the use of viral vectors in patients.

This approach has been used to efficiently transduce TAT-MHC class I antigens into lymphocytes and dendritic cells and expression of the corresponding MHC class I complex on the cell surface Shibagaki et al., (2002) J. Immunol. 168, 2393-401. The transduced dendritic cells were able to induce cytotoxic T lymphocyte activity in vivo resulting in partial tumor regression.

The delivery of therapeutic substances into the central nervous system is severely limited due to the restriction imposed by the blood-brain barrier. Although recently several peptides and proteins have been identified which can prevent neuronal cell death after brain injury in vitro their potential application in vivo is hindered by the inability to deliver them to the site of injury. For instance, the Bcl-2 family member, Bcl-xL, has been previously shown to reduce infarct size following cerebral ischemia in overexpressing transgenic mice Wiessner et al., (1999) Neurosci. Lett. 268, 119-22, however no practical means exists to increase Bcl-xL expression following stroke.

Using TAT fusion technology intraperitoneal administration of TAT-Bcl-xL could prevent apoptotic neuronal cell death following ischemic brain injury Cao et al., (2002) J. Neurosci. 22, 5423-31, Kilic et al., (2002) Ann. Neurol. 52, 617-22, Dietz et al., (2002) Mol. Cell Neurosci. 21, 29-37.

In an elegant approach for treatment of HIV infection a ‘Trojan Horse’ strategy was used to induce cell death in infected cells Vocero-Akbani et al., (1999) Nat. Med. 5, 29-33. While many conventional therapies use drugs to target the HIV protease and block its activity, in this case, the HIV protease present in infected cells was used to activate a killing molecule. By engineering a transducible caspase-3 pro-apoptotic TAT PTD fusion zymogen which substituted HIV proteolytic cleavage sites for endogenous caspase cleavage sites, procasapse-3 was selectively processed into an active protease only in HIV infected cells, resulting in their cell death while uninfected cells were spared. In contrast to protease inhibitor therapies which prolong the longevity of infected cells, this strategy would specifically kill HIV infected cells, resulting in a high therapeutic index for patients. By harnessing the power of TAT transduction to promote the efficient delivery of protein into cells, this approach should be adaptable for in vivo use as a potential anti-HIV therapy. Moreover, a similar approach using other pathogen-encoded proteases could be helpful in preventing infectious diseases such as hepatitis C, cytomegalovirus and malaria.

As used herein, a “fusogenic” domain is any polypeptide that facilitates the destabilization of a cell membrane or the membrane of a cell organelle. For example, the hemagglutinin (HA) of influenza is the major glycoprotein component of the viral envelope. It has a dual function in mediating attachment of the virus to the target cell and fusion of the viral envelope membrane with target cell membranes. In the normal course of viral infection, virus bound to the cell surface is taken up into endosomes and exposed to relatively low pH. The pH change triggers fusion between the viral envelope and the endosomal membrane, as well as conformational changes in HA, which lead to increased exposure of the amino terminus. HA is homotrimeric and is composed of two polypeptide segments, designated HA1 and HA2. The HA1 segments form sialic acid-binding sites and mediate HA attachment to the host cell surface. The HA2 segment forms a membrane-spanning anchor, and its amino-terminal region is involved in a fusion reaction mechanism. Synthetic peptides such as the N-terminus region of the influenza hemagglutinin protein destabilize membranes. Examples of HA2 analogs include GLFGAIAGFIEGGWTGMIDG (SEQ ID NO:2) and GLFEAIAEFIEGGWEGLIEG (SEQ ID NO:3).

Other fusogenic proteins include, for example, the M2 protein of influenza A viruses employed on its own or in combination with the hemagglutinin of influenza virus or with mutants of neuraminidase of influenza A, which lack enzyme activity, but which bring about hemagglutination; peptide analogs of the influenza virus hemagglutinin; the HEF protein of the influenza C virus, the fusion activity of the HEF protein is activated by cleavage of the HEFO into the subunits HEF1 and HEF2; the transmembrane glycoprotein of filoviruses, such as, for example, the Marburg virus, the Ebola virus; the transmembrane glycoprotein of the rabies virus; the transmembrane glycoprotein (G) of the vesicular stomatitis virus; the fusion polypeptide of the Sendai virus, in particular the amino-terminal 33 amino acids of the F1 component; the transmembrane glycoprotein of the Semliki forest virus, in particular the E1 component, the transmembrane glycoprotein of the tickborn encephalitis virus; the fusion polypeptide of the human respiratory syncytial virus (RSV) (in particular the gp37 component); the fusion polypeptide (S protein) of the hepatitis B virus; the fusion polypeptide of the measles virus; the fusion polypeptide of the Newcastle disease virus; the fusion polypeptide of the visna virus; the fusion polypeptide of murine leukemia virus (in particular p15E); the fusion polypeptide of the HTL virus (in particular gp21); and the fusion polypeptide of the simian immunodeficiency virus (SIV). Viral fusogenic proteins are obtained either by dissolving the coat proteins of a virus concentration with the aid of detergents (such as, for example, Y-D-octylglucopyranoside) and separation by centrifugation (review in Mannio et al., BioTechniques 6, 682 (1988)) or else with the aid of molecular biology methods known to the person skilled in the art.

The disclosure provides chimeric/fusion polypeptides comprising a PTD and a heterologous molecule. In one aspect, the chimeric/fusion polypeptide comprises a PTD linked to a heterologous molecule such as a polynucleotide, a small molecule, or a heterologous polypeptide domain. In another aspect, the chimeric/fusion polypeptide comprises a PTD linked to a fusogenic domain.

A polypeptide refers to a polymer in which the monomers are amino acid residues which are joined together through amide bonds. When the amino acids are alpha-amino acids, either the L-optical isomer or the D-optical isomer can be used. A polypeptide encompasses an amino acid sequence and includes modified sequences such as glycoproteins, retro-inverso polypeptides, D-amino acid modified polypeptides, and the like. A polypeptide includes naturally occurring proteins, as well as those which are recombinantly or synthetically synthesized. “Fragments” are a portion of a polypeptide. The term “fragment” refers to a portion of a polypeptide which exhibits at least one useful epitope or functional domain. The term “functional fragment” refers to fragments of a polypeptide that retain an activity of the polypeptide. For example, a functional fragment of a PTD includes a fragment which retains transduction activity. Biologically functional fragments, for example, can vary in size from a polypeptide fragment as small as an epitope capable of binding an antibody molecule, to a large polypeptide capable of participating in the characteristic induction or programming of phenotypic changes within a cell. An “epitope” is a region of a polypeptide capable of binding an immunoglobulin generated in response to contact with an antigen.

In some embodiments, retro-inverso peptides are used. “Retro-inverso” means an amino-carboxy inversion as well as enantiomeric change in one or more amino acids (i.e., levantory (L) to dextrorotary (D)). A polypeptide of the disclosure encompasses, for example, amino-carboxy inversions of the amino acid sequence, amino-carboxy inversions containing one or more D-amino acids, and non-inverted sequence containing one or more D-amino acids. Retro-inverso peptidomimetics that are stable and retain bioactivity can be devised as described by Brugidou et al. (Biochem. Biophys. Res. Comm. 214(2): 685-693, 1995) and Chorev et al. (Trends Biotechnol. 13(10): 438-445, 1995). The overall structural features of a retro-inverso polypeptide are similar to those of the parent L-polypeptide. The two molecules, however, are roughly mirror images because they share inherently chiral secondary structure elements. Main-chain peptidomimetics based on peptide-bond reversal and inversion of chirality represent important structural alterations for peptides and proteins, and are highly significant for biotechnology. Antigenicity and immunogenicity can be achieved by metabolically stable antigens such as all-D- and retro-inverso-isomers of natural antigenic peptides. Several PTD-derived peptidomimetics are provided herein.

Polypeptides and fragments can have the same or substantially the same amino acid sequence as the naturally occurring protein. “Substantially identical” means that an amino acid sequence is largely, but not entirely, the same, but retains a functional activity of the sequence to which it is related. An example of a functional activity is that the fragment is capable of transduction or fusogenic activity. For example, fragments of full length TAT are described herein that have transduction activity. In general two amino acid sequences are “substantially identical” if they are at least 85%, 90%, 95%, 98% or 99% identical, or if there are conservative variations in the sequence. A computer program, such as the BLAST program (Altschul et al., 1990) can be used to compare sequence identity.

In another aspect, the disclosure provides a method of producing a fusion polypeptide comprising a PTD domain and a heterologous molecule or a fusogenic domain by growing a host cell comprising a polynucleotide encoding the fusion polypeptide under conditions that allow expression of the polynucleotide, and recovering the fusion polypeptide. A polynucleotide encoding a fusion polypeptide of the disclosure can be operably linked to a promoter for expression in a prokaryotic or eukaryotic expression system. For example, such a polynucleotide can be incorporated in an expression vector.

Delivery of a polynucleotide of the disclosure can be achieved by introducing the polynucleotide into a cell using a variety of methods known to those of skill in the art. For example, a construct comprising such a polynucleotide can be delivered into a cell using a colloidal dispersion system. Alternatively, a polynucleotide construct can be incorporated (i.e., cloned) into an appropriate vector. For purposes of expression, the polynucleotide encoding a fusion polypeptide of the disclosure may be inserted into a recombinant expression vector. The term “recombinant expression vector” refers to a plasmid, virus, or other vehicle known in the art that has been manipulated by insertion or incorporation of a polynucleotide encoding a fusion polypeptide of the disclosure. The expression vector typically contains an origin of replication, a promoter, as well as specific genes that allow phenotypic selection of the transformed cells. Vectors suitable for such use include, but are not limited to, the T7-based expression vector for expression in bacteria (Rosenberg et al., Gene, 56:125, 1987), the pMSXND expression vector for expression in mammalian cells (Lee and Nathans, J. Biol. Chem., 263:3521, 1988), baculovirus-derived vectors for expression in insect cells, cauliflower mosaic virus, CaMV, and tobacco mosaic virus, TMV, for expression in plants.

Depending on the vector utilized, any of a number of suitable transcription and translation elements (regulatory sequences), including constitutive and inducible promoters, transcription enhancer elements, transcription terminators, and the like may be used in the expression vector (see, e.g., Bitter et al., Methods in Enzymology, 153:516-544, 1987). These elements are well known to one of skill in the art.

The term “operably linked” or “operably associated” refers to functional linkage between the regulatory sequence and the polynucleotide regulated by the regulatory sequence. The operably linked regulatory sequence controls the expression of the product expressed by the polynucleotide.

In yeast, a number of vectors containing constitutive or inducible promoters may be used. (Current Protocols in Molecular Biology, Vol. 2, Ed. Ausubel et al., Greene Publish. Assoc. & Wiley Interscience, Ch. 13, 1988; Grant et al., “Expression and Secretion Vectors for Yeast,” in Methods in Enzymology, Eds. Wu & Grossman, Acad. Press, N.Y., Vol. 153, pp. 516-544, 1987; Glover, DNA Cloning, Vol. II, IRL Press, Wash., D.C., Ch. 3, 1986; “Bitter, Heterologous Gene Expression in Yeast,”Methods in Enzymology, Eds. Berger & Kimmel, Acad. Press, N.Y., Vol. 152, pp. 673-684, 1987; and The Molecular Biology of the Yeast Saccharomyces, Eds. Strathern et al., Cold Spring Harbor Press, Vols. I and II, 1982). A constitutive yeast promoter, such as ADH or LEU2, or an inducible promoter, such as GAL, may be used (“Cloning in Yeast,” Ch. 3, R. Rothstein In: DNA Cloning Vol. 11, A Practical Approach, Ed. DM Glover, IRL Press, Wash., D.C., 1986). Alternatively, vectors may be used which promote integration of foreign DNA sequences into the yeast chromosome.

An expression vector can be used to transform a target cell. By “transformation” is meant a permanent genetic change induced in a cell following incorporation of a polynucleotide exogenous to the cell. Where the cell is a mammalian cell, a permanent genetic change is generally achieved by introduction of the polynucleotide into the genome of the cell. By “transformed cell” is meant a cell into which (or into an ancestor of which) has been introduced, by means of molecular biology techniques, a polynucleotide encoding a fusion polypeptide comprising a PTD linked to a heterologous polypeptide or fusogenic polypeptide. Transformation of a host cell may be carried out by conventional techniques as are known to those skilled in the art. Where the host is prokaryotic, such as E. coli, competent cells which are capable of polynucleotide uptake can be prepared from cells harvested after exponential growth phase and subsequently treated by the CaCl₂ method by procedures well known in the art. Alternatively, MgCl₂ or RbCl can be used. Transformation can also be performed after forming a protoplast of the host cell or by electroporation.

A fusion polypeptide of the disclosure can be produced by expression of polynucleotide encoding a fusion polypeptide in prokaryotes. These include, but are not limited to, microorganisms, such as bacteria transformed with recombinant bacteriophage DNA, plasmid DNA, or cosmid DNA expression vectors encoding a fusion polypeptide of the disclosure. The constructs can be expressed in E. coli in large scale for in vitro assays. Purification from bacteria is simplified when the sequences include tags for one-step purification by nickel-chelate chromatography. Thus, a polynucleotide encoding a fusion polypeptide can also comprise a tag to simplify isolation of the fusion polypeptide. For example, a polyhistidine tag of, e.g., six histidine residues, can be incorporated at the amino terminal end of the fusion polypeptide. The polyhistidine tag allows convenient isolation of the protein in a single step by nickel-chelate chromatography. A fusion polypeptide of the disclosure can also be engineered to contain a cleavage site to aid in protein recovery or other linker moiety separating a PTD from a heterologous molecule. Typically a linker will be a peptide linker moiety. The length of the linker moiety is chosen to optimize the biological activity of the polypeptide comprising PTD domain and a heterologous molecule and can be determined empirically without undue experimentation. The linker moiety should be long enough and flexible enough to allow a PTD polypeptide to freely interact. A linker moiety is a peptide between about one and 30 amino acid residues in length, typically between about two and 15 amino acid residues. Examples of linker moieties are --Gly--Gly--, GGGGS (SEQ ID NO:4), (GGGGS)N (SEQ ID NO:5), GKSSGSGSESKS (SEQ ID NO:6), GSTSGSGKSSEGKG (SEQ ID NO:7), GSTSGSGKSSEGSGSTKG (SEQ ID NO:8), GSTSGSGKPGSGEGSTKG (SEQ ID NO:9), or EGKSSGSGSESKEF (SEQ ID NO:10). Linking moieties are described, for example, in Huston et al., Proc. Nat'l Acad. Sci 85:5879, 1988; Whitlow et al., Protein Engineering 6:989, 1993; and Newton et al., Biochemistry 35:545, 1996. Other suitable peptide linkers are those described in U.S. Pat. Nos. 4,751,180 and 4,935,233, which are hereby incorporated by reference. A DNA sequence encoding a desired peptide linker can be inserted between, and in the same reading frame as, a polynucleotide encoding a PTD polypeptide or fragment thereof followed by a heterologous polypeptide, using any suitable conventional technique. For example, a chemically synthesized oligonucleotide encoding the linker can be ligated between two coding polynucleotides. In particular embodiments, a fusion polypeptide comprises from two to four separate domains (e.g., a PTD domain and a heterologous polypeptide domain) are separated by peptide linkers.

When the host is a eukaryote, such methods of transfection of DNA as calcium phosphate co-precipitates, conventional mechanical procedures, such as microinjection, electroporation, insertion of a plasmid encased in liposomes, or virus vectors may be used. Eukaryotic cells can also be cotransfected with a polynucleotide encoding the PTD-fusion polypeptide of the disclosure, and a second polynucleotide molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein. (Eukaryotic Viral Vectors, Cold Spring Harbor Laboratory, Gluzman ed., 1982).

Eukaryotic systems, and typically mammalian expression systems, allow for proper post-translational modifications of expressed mammalian proteins to occur. Eukaryotic cell s that possess the cellular machinery for proper processing of the primary transcript, glycosylation, phosphorylation, and advantageously secretion of the gene product can be used as host cells for the expression of the PTD-fusion polypeptide of the disclosure. Such host cell lines may include, but are not limited to, CHO, VERO, BHK, HeLa, COS, MDCK, Jurkat, HEK-293, and WI38.

For long-term, high-yield production of recombinant proteins, stable expression is preferred. Rather than using expression vectors that contain viral origins of replication, host cells can be transformed with the cDNA encoding a fusion polypeptide of the disclosure controlled by appropriate expression control elements (e.g., promoter, enhancer, sequences, transcription terminators, polyadenylation sites, and the like), and a selectable marker. The selectable marker in the recombinant plasmid confers resistance to the selection and allows cells to stably integrate the plasmid into their chromosomes and grow to form foci that, in turn, can be cloned and expanded into cell lines. For example, following the introduction of foreign DNA, engineered cells may be allowed to grow for 1-2 days in an enriched media, and then are switched to a selective media. A number of selection systems may be used, including, but not limited to, the herpes simplex virus thymidine kinase (Wigler et al., Cell, 11:223, 1977), hypoxanthine-guanine phosphoribosyltransferase (Szybalska & Szybalski, Proc. Natl. Acad. Sci. USA, 48:2026, 1962), and adenine phosphoribosyltransferase (Lowy et al., Cell, 22:817, 1980) genes can be employed in tk-, hgprt- or aprt- cells, respectively. Also, antimetabolite resistance can be used as the basis of selection for dhfr, which confers resistance to methotrexate (Wigler et al., Proc. Natl. Acad. Sci. USA, 77:3567, 1980; O'Hare et al., Proc. Natl. Acad. Sci. USA, 8:1527, 1981); gpt, which confers resistance to mycophenolic acid (Mulligan & Berg, Proc. Natl. Acad. Sci. USA, 78:2072, 1981; neo, which confers resistance to the aminoglycoside G-418 (Colberre-Garapin et al., J. Mol. Biol., 150:1, 1981); and hygro, which confers resistance to hygromycin genes (Santerre et al., Gene, 30:147, 1984). Additional selectable genes have been described, namely trpB, which allows cells to utilize indole in place of tryptophan; hisD, which allows cells to utilize histinol in place of histidine (Hartman & Mulligan, Proc. Natl. Acad. Sci. USA, 85:8047, 1988); and ODC (ornithine decarboxylase), which confers resistance to the ornithine decarboxylase inhibitor, 2-(difluoromethyl)-DL-ornithine, DFMO (McConlogue L., In: Current Communications in Molecular Biology, Cold Spring Harbor Laboratory, ed., 1987).

Techniques for the isolation and purification of either microbially or eukaryotically expressed PTD-fusion polypeptides of the disclosure may be by any conventional means, such as, for example, preparative chromatographic separations and immunological separations, such as those involving the use of monoclonal or polyclonal antibodies or antigen.

A pharmaceutical composition according to the disclosure can be prepared to include a polypeptide of the disclosure, into a form suitable for administration to a subject using carriers, excipients, and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol, and polyhydric alcohols. Intravenous vehicles include fluid and nutrient replenishers. Preservatives include antimicrobial, anti-oxidants, chelating agents, and inert gases. Other pharmaceutically acceptable carriers include aqueous solutions, non-toxic excipients, including salts, preservatives, buffers and the like, as described, for instance, in Remington's Pharmaceutical Sciences, 15th ed., Easton: Mack Publishing Co., 1405-1412, 1461-1487 (1975), and The National Formulary XIV., 14 th ed., Washington: American Pharmaceutical Association (1975), the contents of which are hereby incorporated by reference. The pH and exact concentration of the various components of the pharmaceutical composition are adjusted according to routine skills in the art. See Goodman and Gilman's, The Pharmacological Basis for Therapeutics (7th ed.).

The pharmaceutical compositions according to the disclosure may be administered locally or systemically. By “therapeutically effective dose” is meant the quantity of a compound according to the disclosure necessary to prevent, to cure, or at least partially arrest the symptoms of tissue damage. Amounts effective for this use will, of course, depend on the severity of the disease and the weight and general state of the patient. Typically, dosages used in vitro may provide useful guidance in the amounts useful for in situ administration of the pharmaceutical composition, and animal models may be used to determine effective dosages for treatment of particular disorders. Various considerations are described, e.g., in Langer, Science, 249: 1527, (1990); Gilman et al. (eds.) (1990), each of which is herein incorporated by reference.

As used herein, “administering a therapeutically effective amount” is intended to include methods of giving or applying a pharmaceutical composition of the disclosure to a subject that allow the composition to perform its intended therapeutic function. The therapeutically effective amounts will vary according to factors, such as the degree of infection in a subject, the age, sex, and weight of the individual. Dosage regima can be adjusted to provide the optimum therapeutic response. For example, several divided doses can be administered daily or the dose can be proportionally reduced as indicated by the exigencies of the therapeutic situation.

The pharmaceutical composition can be administered in a convenient manner, such as by injection (subcutaneous, intravenous, etc.), oral administration, inhalation, transdermal application, or rectal administration. Depending on the route of administration, the pharmaceutical composition can be coated with a material to protect the pharmaceutical composition from the action of enzymes, acids, and other natural conditions that may inactivate the pharmaceutical composition. The pharmaceutical composition can also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersions. In all cases, the composition must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyetheylene glycol, and the like), suitable mixtures thereof, and vegetable oils. The proper fluidity can be maintained, for example, by the use of a coating, such as lecithin, by the maintenance of the required particle size, in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent that delays absorption, for example, aluminum monostearate and gelatin.

Sterile injectable solutions can be prepared by incorporating the pharmaceutical composition in the required amount in an appropriate solvent with one or a combination of ingredients enumerated above, as required, followed by filtered sterilization. Generally, dispersions are prepared by incorporating the pharmaceutical composition into a sterile vehicle that contains a basic dispersion medium and the required other ingredients from those enumerated above.

The pharmaceutical composition can be orally administered, for example, with an inert diluent or an assimilable edible carrier. The pharmaceutical composition and other ingredients can also be enclosed in a hard or soft-shell gelatin capsule, compressed into tablets, or incorporated directly into the individual's diet. For oral therapeutic administration, the pharmaceutical composition can be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations can, of course, be varied and can conveniently be between about 5% to about 80% of the weight of the unit.

The tablets, troches, pills, capsules, and the like can also contain the following: a binder, such as gum gragacanth, acacia, corn starch, or gelatin; excipients such as dicalcium phosphate; a disintegrating agent, such as corn starch, potato starch, alginic acid, and the like; a lubricant, such as magnesium stearate; and a sweetening agent, such as sucrose, lactose or saccharin, or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar, or both. A syrup or elixir can contain the agent, sucrose as a sweetening agent, methyl and propylparabens as preservatives, a dye, and flavoring, such as cherry or orange flavor. Of course, any material used in preparing any dosage unit form should be pharmaceutically pure and substantially non-toxic in the amounts employed. In addition, the pharmaceutical composition can be incorporated into sustained-release preparations and formulations.

Thus, a “pharmaceutically acceptable carrier” is intended to include solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the pharmaceutical composition, use thereof in the therapeutic compositions and methods of treatment is contemplated. Supplementary active compounds can also be incorporated into the compositions.

It is especially advantageous to formulate parenteral compositions in dosage unit form for ease of administration and uniformity of dosage. “Dosage unit form” as used herein, refers to physically discrete units suited as unitary dosages for the individual to be treated; each unit containing a predetermined quantity of pharmaceutical composition is calculated to produce the desired therapeutic effect in association with the required pharmaceutical carrier. The specification for the novel dosage unit forms of the disclosure are dictated by and directly dependent on: (a) the unique characteristics of the pharmaceutical composition and the particular therapeutic effect to be achieve, and (b) the limitations inherent in the art of compounding such an pharmaceutical composition for the treatment of a pathogenic infection in a subject.

The principal pharmaceutical composition is compounded for convenient and effective administration in effective amounts with a suitable pharmaceutically acceptable carrier in an acceptable dosage unit. In the case of compositions containing supplementary active ingredients, the dosages are determined by reference to the usual dose and manner of administration of the said ingredients.

### EXAMPLES

In an effort to exploit TAT-mediated protein delivery developed a bacterial expression system which permitted the rapid cloning and expression of in-frame fusion polypeptides using an N-terminal 11 amino acid sequence corresponding to amino acids 47-57 of TAT has been developed (Nagahara et al., supra; Becker-Hapak et al., Methods 24:247-56, 2001; Schwarze et al., Science 285:1569-72, 1999). In this way, cDNA of the protein of interest is cloned in-frame with the N-terminal 6 ×His-TAT-HA encoding region in the pTAT-HA expression vector. The 6 ×His motif provides for the convenient purification of proteins using metal affinity chromatography and the HA epitope tag allows for immunological analysis of the fusion polypeptide.

Although recombinant proteins can be expressed as soluble proteins within E. coli, TAT-fusion polypeptides are often found within bacterial inclusion bodies. In the latter case, these proteins are extracted from purified inclusion bodies in a relatively pure form by lysis in denaturant, such as in 8 M urea. The denaturation aids in the solubilization of the recombinant protein and assists in the unfolding of complex tertiary protein structure which has been observed to lead to an increase in the transduction efficiency over highly-folded, native proteins (Becker-Hapak et al., supra). This latter observation is in keeping with earlier findings which supported a role for protein unfolding in the increased cellular uptake of the TAT-fusion polypeptide TAT-DHFR (Bonifaci et al., Aids 9:995-1000, 1995). It is thought that the higher energy (DG) partial or fully denatured proteins may transduce more efficiently than lower energy, correctly folded proteins, in part due to increased exposure of the TAT domain. Once inside the cells, these denatured proteins are thought to be correctly refolded by cellular chaperones such as HSP90 in order to regain biological activity (Schneider et al., Proc. Natl. Acad. Sci. USA 93:14536-41, 1996).

Following solubilization, bacterial lysates are incubated with NiNTA resin (Qiagen) which binds to the 6 ×His domain in the recombinant proteins. After washing, these proteins are eluted from the column using increasing concentrations of imidazole. Proteins are further purified using ion exchange chromatography and finally exchanged into PBS +10% glycerol by gel filtration (Nagahara et al., supra).

Purification of TAT-Cre. Cre cDNA was cloned in-frame into the pTAT v2.2 vector that contains an amino-terminal tat-basic domain (48-57) and a carboxy-terminal 6-His tag. TAT-Cre was expressed in BL21 pLysS (Novagen) e.coli. Cultures were grown in Luria broth overnight and induced using 500 mM IPTG for 3 h. Cell pellets were washed and stored at −80° C. until used. TAT-Cre protein was purified in a two step process using metal affinity chromatography (Qiagen) followed by ion exchange using a HiPrep Source 30S 5/5 column (Pharmacia). Aliquots were stored at −80° C. Fluorescent labeling of TAT-Cre was achieved by coupling of the protein to either alexa-488 or alexa-546 protein labeling kits (Molecular Probes).

Cell culture and measurements of recombination. tex.loxP.EG are a murine thymoma cell line that contains an integrated lox-stop-lox eEGFP reporter were maintained in RPMI (Invitrogen) media containing 10% fetal bovine serum (Invitrogen). After treatment with TAT-Cre or control Cre, cells were incubated overnight in complete media and eGFP expression was measured by flow cytometry. Based on propidium iodide exclusion or forward scatter vs. side scatter profile, only live cells were counted. The percentage recombination was calculated by gating on eGFP positive cells. 3T3 loxP.lacZ cells containing a lacZ reporter expressed after recombination were grown in DMEM (Invitrogen) containing 10% fetal bovine serum. Following recombination cells expressing lacZ were assayed by in situ beta-galactosidase staining (Stratagene).

Peptide synthesis. All peptides (HA2-Tat: GLFGAIAGFIENGWEGMIDGGRKKRRQRRR; Tat: GRKKRRQRRR) were synthesized as D-amino acid, retro-inverso forms using solid-phase FMOC chemistry on an Applied Biosystems 431A synthesizer. Peptides were cleaved in 92.5% TFA, 2.5% H₂0, 2.5% thioanisole, 2.5 EDT for 5 h hours, precipitated in ether and purified on C18 reverse phase HPLC column. Major peaks were analyzed by electrospray mass spectrography. Fractions corresponding to the correct molecular weight were lyophilized and stored at −80° C. Prior to use peptides were resuspended in PBS and sterile filtered. The concentration of peptide solutions was determined by absorbance at 215 and 225 nM.

Recombination experiments. To measure the rate of TAT-Cre internalization, tex.loxP.EG cells were plated at 5×10⁵ cells/well and treated with 0.5 μM TAT-Cre in RPMI +/− 10% FBS. After each time period, cells were trypsinized for 2′, washed and replated into complete media overnight. For all drug treatments [0-50 μg/mL chondroitin sulfate A, B, or C (Sigma), 0-25 μg/mL heparin (Sigma), 0-100 μg/mL nystatin (Fluka), 0-5 mM methyl-β-cytochadextrin (Sigma), 0-5 mM amiloride (Sigma) and 0-10 μM cytochalasin D (Sigma)], cells were washed and pretreated for 30′ in serum-free media before the addition of TAT-Cre. Cells were maintained for 1 h in the presence of inhibitors (except for cyclodextrin) aftex which they were washed twice and replated overnight in media containing serum. To measure the effect of nystatin on TAT-Cre internalization, tex.loxP.EG cells were pretreated as described with 10, 25 or 50 μg/mL nystatin for 30′ before the addition of 2 μM TAT-Cre-488 and 4 mM FM4-64. After 1 h, the cells were trypsinized and the fluorescence measured by flow cytometry. To determine the effect of endosomal release by chloroquine, 3T3 loxP.lacZ cells were treated with 0. 25 μM TAT-Cre and 0-200 μM chlorocguine (Sigma) in DMEM +10% FBS overnight. LacZ expression was measured by in situ β-galactosidase staining (Stratagene). For peptide treatments, tex.loxP.EG cells maintained in RPMI +10% FBS were incubated TAT-Cre and either 0-5 mM HA2-tat or tat peptide for 16-20 h after which eGFP expression was measured by flow cytometry.

Fluorescence microscopy. For all imaging experiments cells were grown on chambered glass coverslips (Millipore) To visual TAT-Cre internalization 3T3 cells were incubated with 2 μM fluorescent TAT-Cre 488 and 4 μM FM 4-64. After 8 h, the cells were washed twice with PBS and images were taken using a Nikon epifluoresent microscope. For co-localization, 3T3 cells were transiently transfected with 0.2 μg/well caveolin-1-eGFP expression vector using 0.6 μL Fugene 6 (Roche). After 24 h, cells were washed and incubated with TAT-Cre 546 for 1 h before corresponding fluorescence images were obtained.

Caveolin-1 immunoblot blot. Equal number of cells were solubilized in nonreducing SDS-PAGE sample buffer and resolved on a 12% gel. Proteins were blotted onto PVDF and probed with 1:4000 anti-caveolini pAb (BD-Transduction Laboratories). Bound antibody was detected using 1:5000 anti-rabbit IgG HRP followed by enhanced chemiluminescence (Super Signal, Pierce).

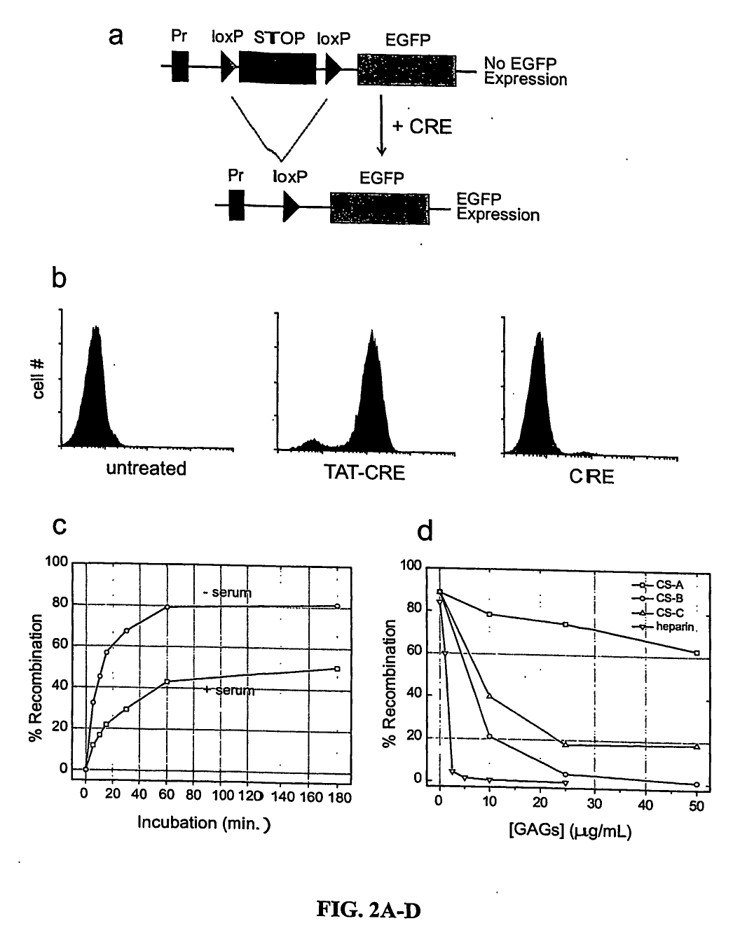

Studies examining internalization of TAT-fusion polypeptides suffered from complications related to cell fixation and visualization. In order to avoid these pitfalls, a TAT-Cre mediated recombination of a lox-stop-lox eGFP reporter gene in live murine T cells (tex.loxP.EG) as a measure for the cellular uptake (FIG. 2a). In this system, exogenous TAT-Cre protein must enter the cell, be translocated to the nucleus and excision the lox-stop-lox DNA segment resulting in GFP expression and measurement 16-20 h later by flow cytometry and microscopy of live cells. Treatment of cells with TAT-Cre resulted in site specific recombination and induction of eGFP expression (FIG. 2b). In contrast, treatment of cells with control Cre protein, expressed and purified under identical conditions, failed to undergo recombination and express eGFP. Thus, expression of eGFP is dependent on transduction of TAT-Cre.

To measure the kinetics of cellular uptake, cells were treated with 0.5 mM TAT-Cre for various amounts of time in the presence and absence of serum. After each time point, cells were washed and trypsinized to remove any surface-bound TAT-Cre. Expression of eGFP increased in relation to the duration of TAT-CRE incubation up to 60′ (FIG. 2c). Surprisingly, exposure of TAT-Cre for, as little as, 5′ was sufficient to induce recombination suggesting that cellular uptake was a rapid process. In addition, tat-cre internalization was temperature sensitive and could be inhibited by incubation of cells at 4° C. Interestingly, both the dose-dependence and kinetics of recombination were negatively affected by the presence of serum in the media (FIG. 2c); however, no degradation of TAT-Cre was detected by immunoblot analysis.

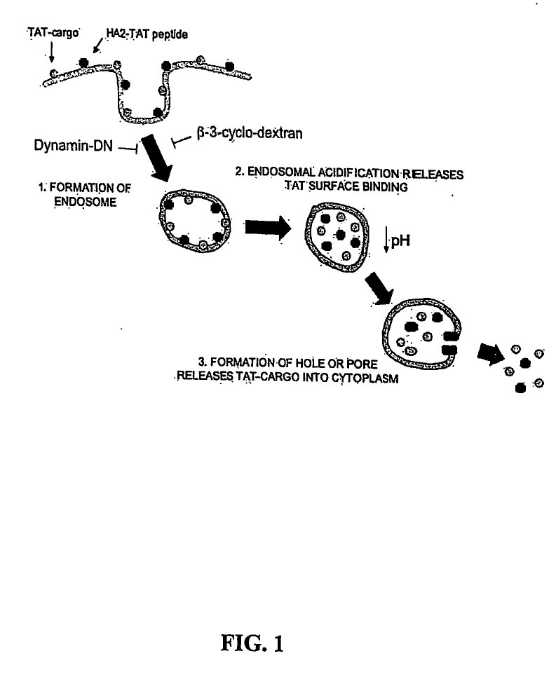

Full-length TAT protein has previously been reported to bind strongly to cell surface heparin sulfate proteoglycans. Incubation of tex.loxP.EG T cells with fluorescently labeled alexa 488 TAT-Cre (TAT-Cre-488) resulted in significant trypsin-sensitive surface binding at 4° C. To determine whether cell surface binding was a necessary and prerequisite step for TAT-Cre internalization, cells were incubated with TAT-Cre and increasing concentrations of free glycosaminglyans for 1 hr in serum-free media, then washed and replated the cells in complete media, and measured eGFP expression after 16 hr. Chondroitin sulfates B and C and heparin prevented surface binding of TAT-Cre and strongly inhibited subsequent recombination (FIG. 1d). These results indicated that presumably electrostatic interactions between the cationic TAT-domain and the cell surface is a necessary event prior to internalization (FIG. 1d).

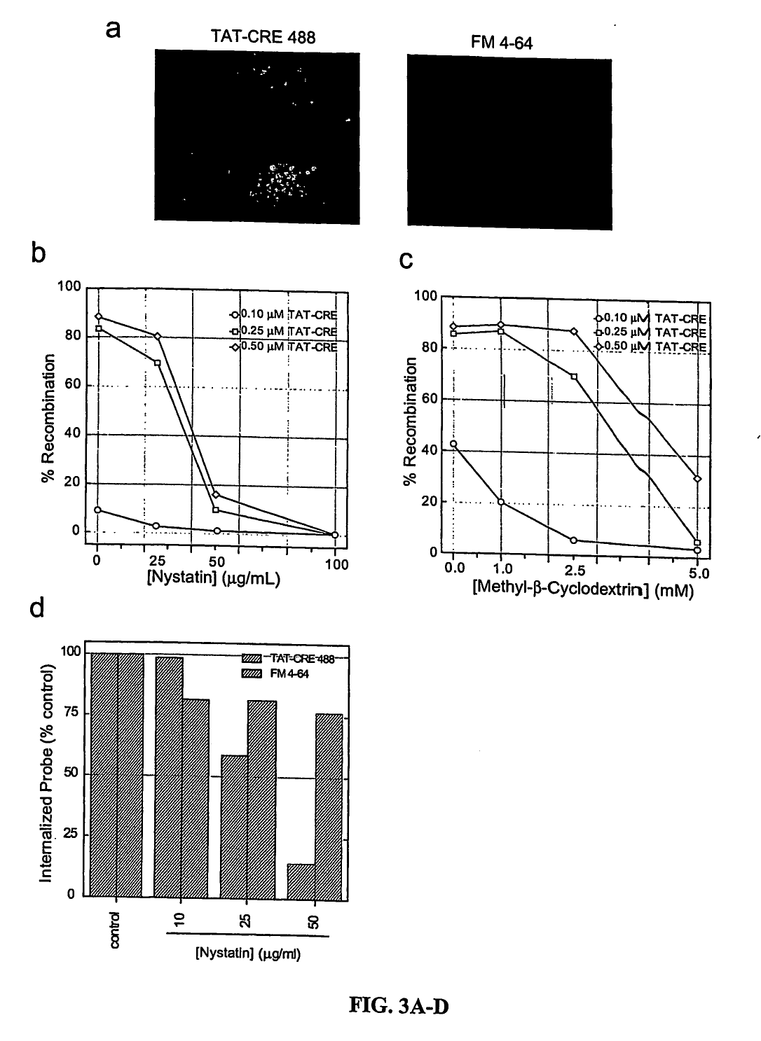

Endocytosis is an essential mechanism for the internalization of a variety of extracellular factors 11. Recently several studies have shown that the uptake of native TAT protein and recombinant TAT-fusion polypeptides occurs by endocytosis. Similarly, fluorescently labeled TAT-Cre-488 was internalized and co-localized with FM4-64, a general fluorescent marker of endocytosis, in live NIH-3T3 cells (FIG. 3a). Given that endocytosis occurs by variety of mechanisms and that TAT-Cre has a high electrostatic avidity for the cell surface, experiments were performed to determine whether cellular uptake of TAT-Cre occurred through a specific endocytotic pathway or by all forms of endocytosis.

The initial focus was on lipid rafts, cholesterol and sphingolipid enriched microdomains in the plasma membrane, which are involved in several endocytic pathways, including caveolin-mediated endocytosis and macropinocytosis. Removal of cholesterol from the plasma membrane disrupts lipid rafts and prevents lipid raft-mediated endocytosis. To determine the involvement of lipid rafts in TAT-Cre endocytosis, cells were pretreated with β-cyclodextrin or nystatin, to deplete or sequester cholesterol, respectively, and then added TAT-Cre for an additional 1 h after which, the cells were trypsinization and replating in complete media overnight. Surprisingly, both β-cyclodextrin and nystatin disruption of lipid rafts resulted in a dose-dependent inhibition of recombination (FIG. 3b, c). To control for inhibition of all forms of endocytosis, cells were co-treated with labeled TAT-Cre-488 protein and the FM4-64 endosornal dye. Importantly, nystatin treatment of cells caused a near complete loss of TAT-Cre-488 internalization, whereas FM4-64 showed only a minor decrease (FIG. 3d). Taken together, these observations demonstrate that lipid raft disruption specifically prevents recombination by limiting the entry of TAT-Cre into cells.

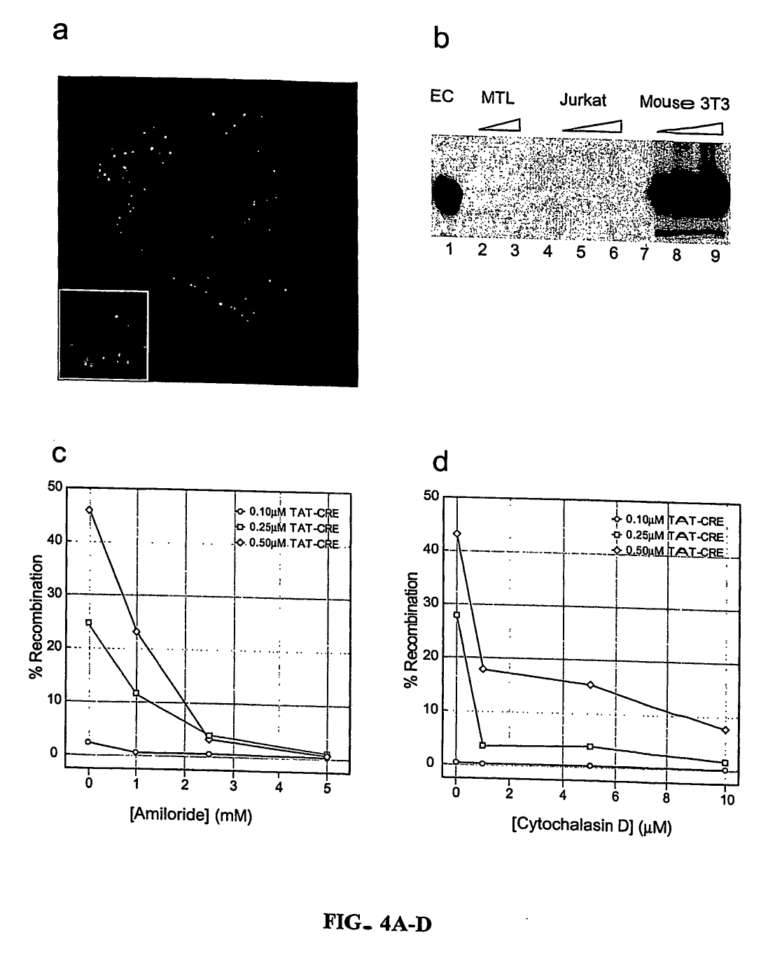

One mechanism of lipid raft-mediated endocytosis is through caveolae, flask shaped invaginations of the plasma membrane involved in the slow transcellular trafficking of serum proteins across endothelial cells. Caveolar-mediated endocytosis is an attractive pathway for TAT-protein internalization because these vesicles do not lead to lysosomes, but are trafficked to an intracellular perinuclear compartment, the caveosome, from where the cargo is further sorted to the endoplasmic reticulum and other cellular locations. It has been suggested that endocytosis of TAT-eGFP fusion polypeptide occurs through caveolar uptake. Therefore, both murine T lymphocytes used here and Jurkat T cells used by Fittipaldi et al. were for caveolin expression. Caveolin expression was not detected in both of these cell lines by immunoblot analysis, whereas endothelial cells and NIH 3T3 cells expressed high levels (FIG. 4a). Moreover, transfection of NIH 3T3 cells with caveolin-1-eGFP also failed to result in co-localization with fluorescently labeled TAT-Cre-546 protein (FIG. 4b), indicating that transduction of TAT-Cre into cells occurs in a lipid raft-dependent, but caveolae-independent manner.

Macropinocytosis is a non-selective, receptor-independent endocytic pathway that has been associated with lipid rafts and is often triggered by stimulation at the cell surface leading to the formation of actin-dependent membrane protrusions that envelope into large vesicles known as macropinosomes. To determine whether macropinocytosis was involved in transduction, cells were pretreated with amiloride, a specific inhibitor of Na+/H+ exchange required for macropinocytosis, or cytochalasin D, which prevents F-actin elongation, for 30 min followed by a 1 hr TAT-Cre treatment, washing, trypsinization and replating in complete media overnight (FIG. 4c,d). Treatment of cells with both compounds resulted in a dose-dependent inhibition of TAT-Cre transduction into the cells and lack of recombination. Taken together with the ability of TAT-Cre to transduce into non-caveolin expressing cells, the large vesicle size and rapid uptake (FIG. 3a), along with TAT-mediated transduction of large cargo sizes (iron beads and liposomes), these observations suggest that the TAT-mediated transduction occurs by lipid raft-mediated macropinocytosis.

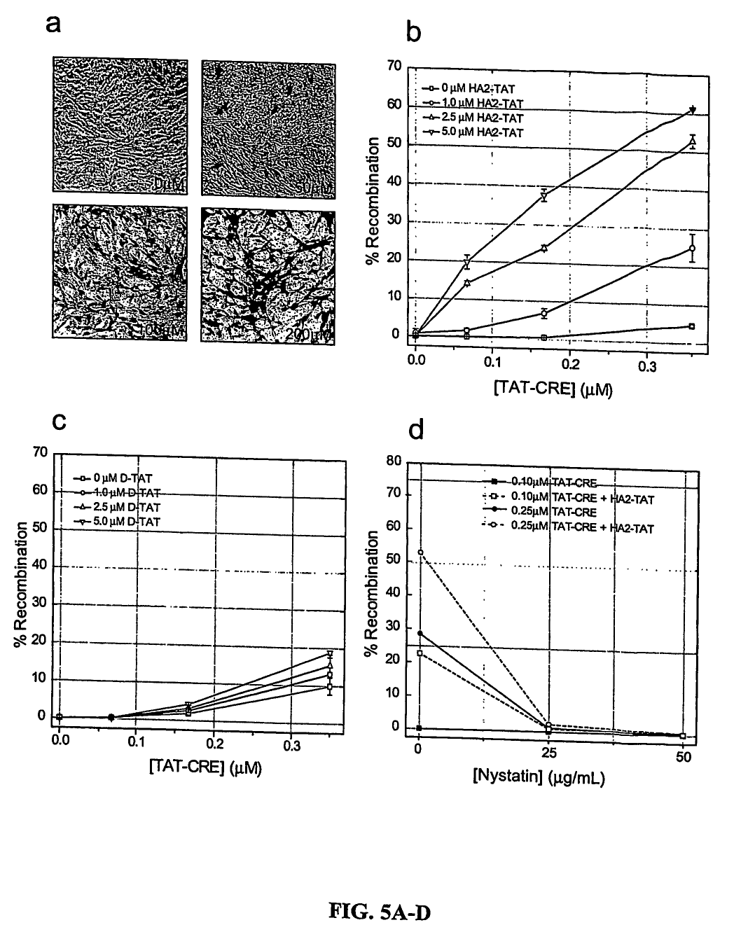

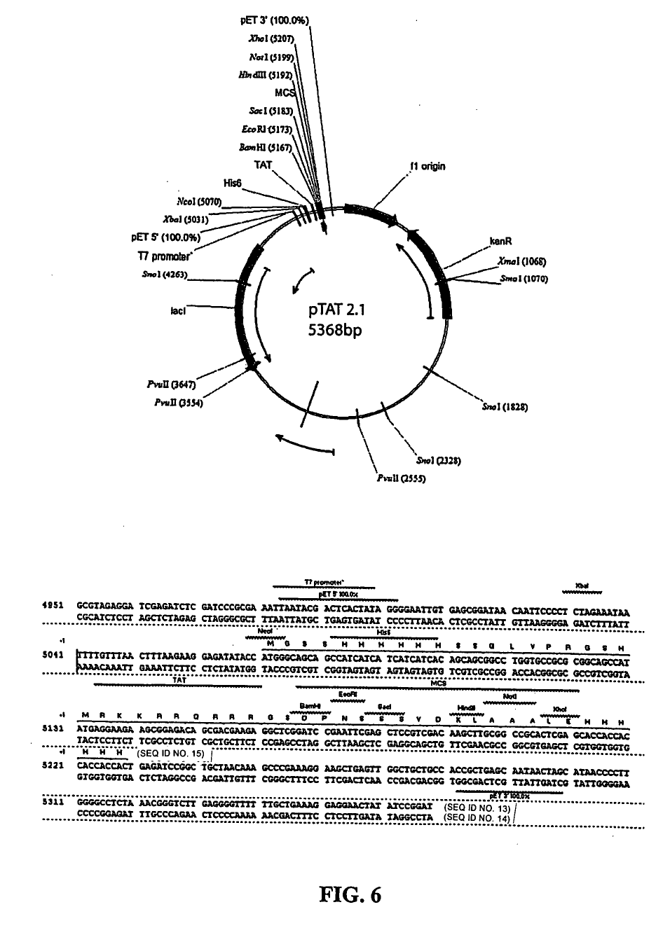

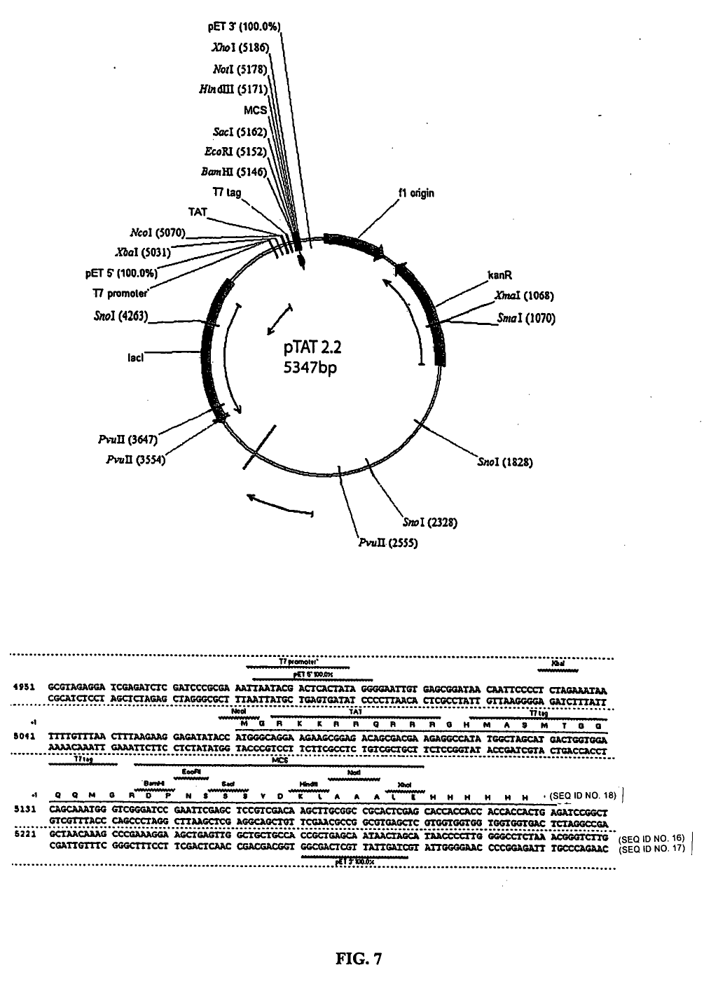

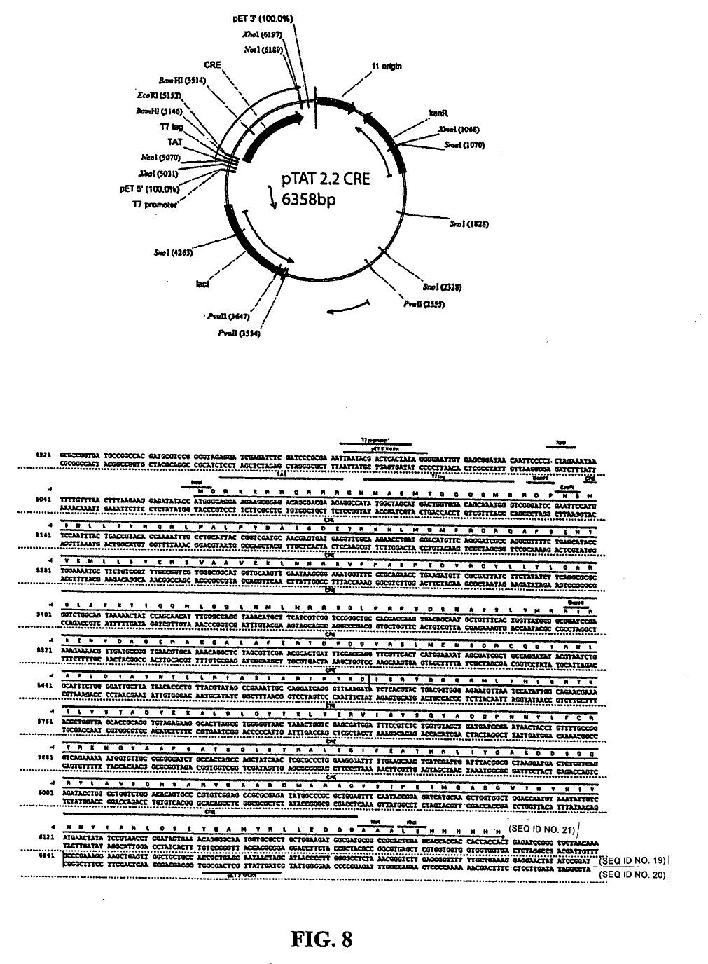

To recombine DNA and induce eGFP expression, TAT-Cre must escape from macropinosomes. However, fluorescent imaging of 3T3 cells treated with TAT-Cre-488 indicated that the majority of protein remained in vesicle-bound compartments after 8 hr (FIG. 3a), indicating that the release of TAT-Cre from macropinosomes was an inefficient process. Therefore to enhance release from macropinosomes, 3T3 LacZ reporter cells were treated with a sub-threshold dose of TAT-Cre in combination with increasing concentrations of chloroquine, an ion-transporting ATPase inhibitor that prevents vesicle acidification leading to endosomal disruption (FIG. 5a). Sub-threshold treatment with TAT-Cre alone did not result in recombination and expression of LacZ. In contrast, addition of 100 μM and 200 μM chloroquine with TAT-Cre caused a significant increase in recombination and LacZ expression (FIG. 5a). However, as shown by the significant loss of cells in chloroquine treated cells (FIG. 5a, bottom right panel), the effective dose of chloroquine resulted in extensive cytotoxicity to multiple several cell lines. So, while demonstrating the potential benefit by stimulating endosomal escape, cytotoxicity associated with a general endosomal disrupter, such as chloroquine, precludes its biological usefulness.

Several viruses have evolved endosomal escape mechanisms to enter the cytoplasm by taking advantage of the vesicle low pH to induce protein confirmational changes that trigger endosomal membrane destabilization 24. The N-terminal 20 amino acids of the influenza virus hemagglutinin (HA) protein, termed HA-2 (GLFGAIAGFIENGWEGMIDG), is a well characterized fusogenic peptide that has been shown to destabilize membranes at low pH. To increase the efficiency of TAT-fusion polypeptide release from macropinosomes, a proteolytically-stable, retro-inverso D-amino acid peptide corresponding to the HA-2 domain peptide followed by the TAT transduction domain (HA2-TAT) was synthesized. Treatment of tex.loxP.EG T cells with a sub-threshold concentration of TAT-Cre protein resulted in minimal recombination and expression of eGFP (FIG. 5b). In contrast, treatment of cells with TAT-Cre and in combination with HA2-TAT peptide resulted in marked increases (>10-fold) in recombination. This enhanced effect appears unrelated to the TAT domain, as cells treated with control TAT D-isomer peptide showed only minor increases in recombination (FIG. 5c). Consistent with the lipid raft-dependent results above, pretreatment with nystatin inhibited HA2-TAT-mediated enhancement of recombination by TAT-Cre (FIG. 5d). Taken together, these observations demonstrate the ability to further enhance TAT-mediated transduction into the cells.

A number of embodiments have been described. Nevertheless, it will be understood that various modifications may be made without departing from the spirit and scope of the description. Accordingly, other embodiments are within the scope of the following claims.

Sequences:

| SEQ ID NO:1 | |
|---|---|
| 1 | mepvdprlep wkhpgsqpkt actncyckkc cfhcqvcfit kalgisygrk krrqrrrppq |
| 61 | gsqthqvsls kqptsqsrgd ptgpke |

What is claimed is

1. A composition comprising:
a) a first fusion polypeptide comprising:
i) a first domain comprising a protein transduction moiety, the transduction moiety comprising a membrane transport function; and
ii) a second domain comprising a heterologous polypeptide;
b) a second fusion polypeptide comprising:
i) a first domain comprising a protein transduction moiety, the transduction moiety comprising a membrane transport function; and
ii) a second domain comprising a fusogenic polypeptide.

2. The composition of claim 1, wherein the protein transduction moiety is selected from the group consisting of a polypeptide comprising a herpesviral VP22 protein; a polypeptide comprising a human immunodeficiency virus (HIV) TAT protein; a polypeptide comprising a homeodomain of an Antennapedia protein (Antp HD), and functional fragments thereof.

3. The composition of claim 2, wherein a TAT protein functional fragment comprises SEQ ID NO:l from amino acid 47-57.

4. The composition of claim 1, wherein the heterologous polypeptide is a therapeutic or diagnostic polypeptide.

5. The composition of claim 4, wherein the diagnostic polypeptide is an imaging agent.

6. The composition of claim 4, wherein the therapeutic polypeptide modulates cell proliferation.

7. The composition of claim 6, wherein the modulation inhibits cell proliferation.

8. The composition of claim 7, wherein the therapeutic agent is a suicide inhibitor or a tumor suppressor protein.

9. The composition of claim 8, wherein the suicide inhibitor is thymidine kinase.

10. The composition of claim 8, wherein the tumor suppressor protein is p53.

11. The composition of claim 6, wherein the modulation increases cell proliferation.

12. The composition of claim 11, wherein the therapeutic agent is selected from the group consisting of SV40 small T antigen, SV40 large T antigen, adenovirus E1A, papilloma virus E6, papilloma virus E7, Epstein-Barr virus, Epstein-Barr nuclear antigen-2, human T-cell leukemia virus-1 (HTLV-1), HTLV-1 tax, herpesvirus saimiri, mutant p53, myc, c-jun, c-ras, c-Ha-ras, h-ras, v-src, c-fgr, myb, c-myc, n-mye, v-myc, and Mdm2.

13. The composition of claim 1, wherein the fusogenic polypeptide is selected from the group consisting of the M2 protein of influenza A viruses; peptide analogs of the influenza virus hemagglutinin; the HEF protein of the influenza C virus; the transmembrane glycoprotein of filoviruses; the transmembrane glycoprotein of the rabies virus; the transmembrane glycoprotein (G) of the vesicular stomatitis virus; the fusion polypeptide of the Sendai virus; the transmembrane glycoprotein of the Semliki forest virus; the fusion polypeptide of the human respiratory syncytial virus (RSV); the fusion polypeptide of the measles virus; the fusion polypeptide of the Newcastle disease virus; the fusion polypeptide of the visna virus; the fusion polypeptide of murine leukemia virus; the fusion polypeptide of the HTL virus; and the fusion polypeptide of the simian immunodeficiency virus (SIV).

14. The composition of claim 1, wherein the fusogenic polypeptide comprises a sequence selected from SEQ ID NO:2 and SEQ ID NO:3.

15. A pharmaceutical or diagnostic composition comprising the composition of claim 1.

16. A kit comprising a vessel or vessels containing
a) a first fusion polypeptide comprising:
i) a first domain comprising a protein transduction moiety, the transduction moiety comprising a membrane transport function; and
ii) a second domain comprising a heterologous polypeptide; and
b) a second fusion polypeptide comprising:
i) a first domain comprising a protein transduction moiety, the transduction moiety comprising a membrane transport function; and
ii) a second domain comprising a fusogenic polypeptide.

17. An article of manufacture comprising a vessel containing
a) a first fusion polypeptide comprising:
i) a first domain comprising a protein transduction moiety, the transduction moiety comprising a membrane transport function; and
ii) a second domain comprising a heterologous polypeptide; and
b) a second fusion polypeptide comprising:
i) a first domain comprising a protein transduction moiety, the transduction moiety comprising a membrane transport function; and
ii) a second domain comprising a fusogenic polypeptide; or
c) packaged together, a vessel containing the polypeptide of a) and a vessel containing the polypeptide of b).

18. An article of manufacture comprising, packaged together:
a) a vessel containing the composition of claim 1; and
b) instructions for use of the composition in a therapeutic or diagnostic method.

19. An article of manufacture comprising, packaged together:
a) a vessel containing a first fusion polypeptide comprising:
i) a first domain comprising a protein transduction moiety, the transduction moiety comprising a membrane transport function; and
ii) a second domain comprising a heterologous polypeptide;
b) a vessel containing a second fusion polypeptide comprising:
i) a first domain comprising a protein transduction moiety, the transduction moiety comprising a membrane transport function; and
ii) a second domain comprising a fusogenic polypeptide; and
c) instructions for use of the polypeptides of a) and b) in a therapeutic or diagnostic method.

20. A method of introducing a heterologous polypeptide in to a target cell, the method comprising contacting the cell with the composition of claim 1.

21. A method of introducing a heterologous polypeptide into a target cell, the method comprising contacting the cell with a composition comprising:
a) a first polypeptide comprising at least one transducing domain associated with a heterologous polypeptide; and
b) a second polypeptide comprising at least one transducing domain associated with a fusogenic domain, wherein the first polypeptide and second polypeptide are co-transduced in to the cell.

22. The method of claim 21, wherein the protein transducing domain is selected from the group consisting of a polypeptide comprising a herpesviral VP22 protein; a polypeptide comprising a human immunodeficiency virus (HIV) TAT protein or a functional fragment thereof; and a polypeptide comprising a homeodomain of an Antennapedia protein (Antp HD).

23. The method of claim 22, wherein a TAT protein functional fragment comprises SEQ ID NO:1 from amino acid 47-57.

24. The method of claim 21, wherein the heterologous polypeptide is a therapeutic or diagnostic polypeptide.

25. The method of claim 24, wherein the diagnostic polypeptide is an imaging agent.

26. The method of claim 24, wherein the therapeutic polypeptide is a suicide inhibitor or a tumor suppressor protein.

27. The method of claim 26, wherein the suicide inhibitor is thymidine kinase.

28. The method of claim 21, wherein the contacting is in vivo or in vitro.

29. The composition of claim 21, wherein the fusogenic polypeptide is selected from the group consisting of the M2 protein of influenza A viruses; peptide analogs of the influenza virus hemagglutinin; the HEF protein of the influenza C virus; the transmembrane glycoprotein of filoviruses; the transmembrane glycoprotein of the rabies virus; the transmembrane glycoprotein (G) of the vesicular stomatitis virus; the fusion polypeptide of the Sendai virus; the transmembrane glycoprotein of the Semliki forest virus; the fusion polypeptide of the human respiratory syncytial virus (RSV); the fusion polypeptide of the measles virus; the fusion polypeptide of the Newcastle disease virus; the fusion polypeptide of the visna virus; the fusion polypeptide of murine leukemia virus; the fusion polypeptide of the HTL virus; and the fusion polypeptide of the simian immunodeficiency virus (SIV).

30. The composition of claim 21, wherein the fusogenic polypeptide comprises a sequence selected from SEQ ID NO:2 and SEQ ID NO:3.

31. A fusion polypeptide comprising a protein transduction domain and a fusogenic domain.

32. The fusion polypeptide of claim 31, wherein the protein transduction moiety is selected from the group consisting of a polypeptide comprising a herpesviral VP22 protein; a polypeptide comprising a human immunodeficiency virus (HIV) TAT protein; a polypeptide comprising a homeodomain of an Antennapedia protein (Antp HD), and functional fragments thereof.

33. The fusion polypeptide of claim 32, wherein a TAT protein functional fragment comprises SEQ ID NO:1 from amino acid 47-57.

34. The fusion polypeptide of claim 31, wherein the fusogenic polypeptide is selected from the group consisting of the M2 protein of influenza A viruses; peptide analogs of the influenza virus hemagglutinin; the HEF protein of the influenza C virus; the transmembrane glycoprotein of filoviruses; the transmembrane glycoprotein of the rabies virus; the transmembrane glycoprotein (G) of the vesicular stomatitis virus; the fusion polypeptide of the Sendai virus; the transmembrane glycoprotein of the Sernliki forest virus; the fusion polypeptide of the human respiratory syncytial virus (RSV); the fusion polypeptide of the measles virus; the fusion polypeptide of the Newcastle disease virus; the fusion polypeptide of the visna virus; the fusion polypeptide of murine leukemia virus; the fusion polypeptide of the HTL virus; and the fusion polypeptide of the simian immunodeficiency virus (SIV).

35. The fusion polypeptide of claim 31, wherein the fusogenic polypeptide comprises a sequence selected from SEQ ID NO:2 and SEQ ID NO:3.


SEQUENCE LISTING (21 sequences)

SEQ ID NO: 1 - protein, 86 aa, Unknown
  transducing protein
MEPVDPRLEPWKHPGSQPKTACTNCYCKKCCFHCQVCFITKALGISYGRKKRRQRRRPPQGSQTHQVSLSKQPTSQSRGDPTGPKE

SEQ ID NO: 2 - protein, 20 aa, Unknown
  HA2 analog
GLFGAIAGFIEGGWTGMIDG

SEQ ID NO: 3 - protein, 20 aa, Unknown
  HA2 analog
GLFEAIAEFIEGGWEGLIEG

SEQ ID NO: 4 - protein, 5 aa, Unknown
  linker moiety
GGGGS

SEQ ID NO: 5 - protein, 6 aa, Unknown
  linker moiety
GGGGSX

SEQ ID NO: 6 - protein, 12 aa, Unknown
  linker moiety
GKSSGSGSESKS

SEQ ID NO: 7 - protein, 14 aa, Unknown
  linker moiety
GSTSGSGKSSEGKG

SEQ ID NO: 8 - protein, 18 aa, Unknown
  linker moiety
GSTSGSGKSSEGSGSTKG

SEQ ID NO: 9 - protein, 18 aa, Unknown
  linker moiety
GSTSGSGKPGSGEGSTKG

SEQ ID NO: 10 - protein, 14 aa, Unknown
  linker moiety
EGKSSGSGSESKEF

SEQ ID NO: 11 - protein, 30 aa, Unknown
  HA2 TAT peptide
GLFGAIAGFIENGWEGMIDGGRKKRRQRRR

SEQ ID NO: 12 - protein, 20 aa, Unknown
  HA2 peptide
GLFGAIAGFIENGWEGMIDG

SEQ ID NO: 13 - DNA, 418 bp, Unknown
  cDNA
gcgtagaggatcgagatctcgatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatgaggaagaagcggagacagcgacgaagaggctcggatccgaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 14 - DNA, 418 bp, Unknown
  cDNA
cgcatctcctagctctagagctagggcgctttaattatgctgagtgatatccccttaacactcgcctattgttaaggggagatctttattaaaacaaattgaaattcttcctctatatggtacccgtcgtcggtagtagtagtagtagtgtcgtcgccggaccacggcgcgccgtcggtatactccttcttcgcctctgtcgctgcttctccgagcctaggcttaagctcgaggcagctgttcgaacgccggcgtgagctcgtggtggtggtggtggtgactctaggccgacgattgtttcgggctttccttcgactcaaccgacgacggtggcgactcgttattgatcgtattggggaaccccggagatttgcccagaactccccaaaaaacgactttcctccttgatataggccta

SEQ ID NO: 15 - protein, 53 aa, Unknown
  TAT peptide
MGSSHHHHHHSSGLVPRGSHMRKKRRQRRRGSDPNSSSVDKLAAALEHHHHHH

SEQ ID NO: 16 - DNA, 360 bp, Unknown
  cDNA
gcgtagaggatcgagatctcgatcccgcgaaattaatacgactcactataggggaattgtgagcggataacaattcccctctagaaataattttgtttaactttaagaaggagatataccatgggcaggaagaagcggagacagcgacgaagaggccatatggctagcatgactggtggacagcaaatgggtcgggatccgaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttg

SEQ ID NO: 17 - DNA, 360 bp, Unknown
  cDNA
cgcatctcctagctctagagctagggcgctttaattatgctgagtgatatccccttaacactcgcctattgttaaggggagatctttattaaaacaaattgaaattcttcctctatatggtacccgtccttcttcgcctctgtcgctgcttctccggtataccgatcgtactgaccacctgtcgtttacccagccctaggcttaagctcgaggcagctgttcgaacgccggcgtgagctcgtggtggtggtggtggtgactctaggccgacgattgtttcgggctttccttcgactcaaccgacgacggtggcgactcgttattgatcgtattggggaaccccggagatttgcccagaac

SEQ ID NO: 18 - protein, 46 aa, Unknown
  TAT peptide
MGRKKRRQRRRGHMASMTGGQQMGRDPNSSSVDKLAAALEHHHHHH

SEQ ID NO: 19 - DNA, 1437 bp, Unknown
  cDNA
gcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcaggaagaagcggagacagcgacgaagaggccatatggctagcatgactggtggacagcaaatgggtcgggatccgaattccatgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgatgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 20 - DNA, 1438 bp, Unknown
  cDNA
cgcggccactacggccggtgctacgcaggccgcatctcctagctctagagctagggcgctttaattatgctgagtgatatccccttaacactcgcctattgttaaggggagatctttattaaaacaaattgaaattcttcctctatatggtacccgtccttcttcgcctctgtcgctgcttctccggtataccgatcgtactgaccacctgtcgtttacccagccctaggcttaaggtacaggttaaatgactggcatgtggttttaaacggacgtaatggccagctacgttgctcactactccaagcgttcttggactacctgtacaagtccctagcggtccgcaaaagactcgtatggaccttttacgaagacaggcaaacggccagcacccgccgtaccacgttcaacttattggcctttaccaaagggcgtcttggacttctacaagcgctaatagaagatatagaactccgcgcgccagaccgtcatttttgataggtcgttgtaaacccggtcgatttgtacgaagtagcagccaggcccgacggtgctggttcactgtcgttacgacaaagtgaccaatacgccgcctaggcttttcttttgcaactacggccacttgcacgttttgtccgagatcgcaagcttgcgtgactaaagctggtccaagcaagtgagtaccttttatcgctagcgacggtcctatatgcattagaccgtaaagacccctaacgaatattgtgggacaatgcatatcggctttaacggtcctagtcccaatttctatagagtgcatgactgccaccctcttacaattaggtataaccgtcttgcttttgcgaccaatcgtggcgtccacatctcttccgtgaatcggacccccattgatttgaccagctcgctacctaaaggcagagaccacatcgactactaggcttattgatggacaaaacggcccagtcttttttaccacaacggcgcggtagacggtggtcggtcgatagttgagcgcgggaccttccctaaaaacttcgttgagtagctaactaaatgccgcgattcctactgagaccagtctctatggaccggaccagacctgtgtcacgggcacagcctcggcgcgctctataccgggcgcgacctcaaagttatggcctctagtacgttcgaccaccgacctggttacatttataacagtacttgatataggcattggacctatcactttgtccccgttaccacgcggacgaccttctaccgctacgccggcgtgagctcgtggtggtggtggtggtgactctaggccgacgattgtttcgggctttccttcgactcaaccgacgacggtggcgactcgttattgatcgtattggggaaccccggagatttgcccagaactccccaaaaaacgactttcctccttgatataggccta

SEQ ID NO: 21 - protein, 383 aa, Unknown
  TAT peptide
MGRKKRRQRRRGHMASMTGGQQMGRDPNSMSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGDAAALEHHHHHH